(19) United States
(12) Patent Application Publication
(10) Pub. No.: US 2024/0148879 A1
(43) Pub. Date: May 9, 2024

(54) GLP-1R, GIP-R AND/OR GCGR AGONISTS, FORMULATIONS, AND METHODS OF USE

(71) Applicant: Spitfire Pharma LLC, Gaithersburg, MD (US)

(72) Inventors: M. Scot Roberts, Myersville, MD (US); Bertrand Victor Gilbert Georges, London (GB)

(21) Appl. No.: 18/547,160

(22) PCT Filed: Feb. 21, 2022
(86) PCT No.: PCT/US22/17175

§ 371 Date: Aug. 20, 2023

Related U.S. Application Data
(60) Provisional application No. 63/151,765, filed on Feb. 21, 2021.

(51) Int. Cl. A61K 47/54; A61K 9/00; A61K 38/00; A61P 3/04; C07K 14/605

(52) U.S. Cl. CPC A61K 47/549; A61K 9/0019; A61P 3/04; C07K 14/605; A61K 38/00

(57) ABSTRACT

This disclosure relates to the field of peptides, formulations, and methods of using the same, including but not limited to modified peptides derived from any of SEQ ID NOS. 1-29.

### RELATED APPLICATIONS

This application claims priority to U.S. Provisional Application Ser. No. 63/151,765 filed on 21 Feb. 2021, the entirety of its contents incorporated herein by reference.

### FIELD OF THE DISCLOSURE

This disclosure relates to the field of GLP-1R, GIP-R and GCGR agonists, formulations, and methods of using the same.

### BACKGROUND OF THE DISCLOSURE

The increasing prevalence of obesity, diabetes mellitus, non-alcoholic fatty liver disease (NAFLD) and its advanced form, non-alcoholic steatohepatitis (NASH), is a world health crisis of epidemic proportions that is a major contributor to patient morbidity and mortality as well as a major economic burden. Obesity is an important risk factor for type 2 diabetes and NASH, and roughly 90% of patients with type 2 diabetes are overweight or obese. Obesity is a rapidly increasing problem worldwide and currently more than 65% of adults in the U.S. are overweight (Hedley, A. A., et al. (2004) JAMA 291: 2847-2850). NASH is anticipated to be the leading cause of liver transplant in the near future. There is a need for development of safe and efficacious pharmaceutical treatments for obesity and diabetes mellitus. The disclosure provides improved peptide pharmaceuticals for treatment of disorders associated with obesity or/and diabetes, such as non-alcoholic steatohepatitis (NASH) and polycystic ovary syndrome (PCOS).

In the United States (US), NASH has become the leading cause of end-stage liver disease or liver transplantation. Obesity is the core driver of NASH and weight loss results in reduction in liver fat and NASH improvement. More than 80% of individuals with NASH are overweight or obese, and with no currently available US Food and Drug Administration (FDA)-approved pharmacologic options for inducing weight loss, therapy has largely been based on lifestyle interventions directed at achieving weight loss. However, it is difficult to attain and maintain long-term weight loss with lifestyle changes alone.

Glucagon-like peptide-1 receptor agonists (GLP-1RA) are associated with modest degrees of weight loss at approved doses, and these agents have emerged as a treatment option for patients with NASH. In a recent clinical trial, liraglutide, a daily GLP-1RA, was associated with resolution of NASH, with a trend towards improvement of liver fibrosis. However, patients lost only 5.5% body weight. In one study, 10% or greater weight loss was required for optimal NASH resolution. Higher levels of weight loss have also been associated with lower incidences of cardiovascular disease and non-hepatic malignancies, which represent the most serious co-morbidities facing NASH patients.

GLP-1RAs exert central effects on appetite and food intake, while GCR agonists drive increased energy expenditure in animal models and humans. The effects of GCR agonist and GLP-1RA have been shown to be synergistic in driving greater degrees of weight loss compared to a GLP-1RA alone. GCRs also enhance lipolysis and suppress liver fat synthesis, providing an additional pathway for liver fat reduction and NASH resolution.

Dual agonists combine GCR with GLP-1RA in the same molecule. In obese non-human primates, chronic administration of a GLP-1R/GCR dual agonist reduced body weight and improved glucose tolerance to a greater degree compared to a GLP-1RA mono-agonist. Clinical studies of cotadutide, a GLP-1/GCR dual agonist with a 5:1 bias of GLP-1 to glucagon activity, demonstrated an impressive 39% reduction in liver fat content in just 6 weeks and greater improvement in NASH-related alanine aminotransferase (ALT) reduction than liraglutide alone. However, the degree of weight loss over 26 weeks of cotadutide administration was comparable to liraglutide (5.4% vs. 5.5%), suggesting that the 5:1 ratio was acceptable for liver fat reduction but suboptimal for weight reduction. Balanced (1:1) agonism has been shown to be associated with greater weight loss and metabolic effects than biased ratios that favor one agonist over the other. A recent study with JNJ 64565111, a balanced dual agonist, achieved an impressive 8% reduction in body weight in just 12 weeks (NCT03586830).

Unfortunately, GLP-1RAs have been associated with high rates of nausea, vomiting and diarrhea. These agents must also be titrated over prolonged periods to reduce side effects, and agents with improved tolerability and dosing regimens are needed. Accordingly, there remains a need for convenient dosing (e.g., weekly instead of daily) with a therapeutic dose to control blood glucose and/or induce weight loss that does not need to be titrated to reach a therapeutic level in the absence of gastrointestinal side effects.

### SUMMARY OF THE DISCLOSURE

Described herein are modified peptides and products thereof (e.g., formulations) and uses of the same for treating disorders associated with the function of glucagon-like peptide 1 receptor (GLP-1R), gastric inhibitory polypeptide receptor (GIP-R) and/or glucagon receptor (GCGR), including but not limited to insulin resistance or/and obesity (including chronic weight management), such as type 2 diabetes, metabolic syndrome, cardiovascular diseases (including coronary artery diseases such as atherosclerosis and myocardial infarction), hypertension, NASH, chronic kidney disease and PCOS, neuronal dysfunction such as Alzheimer's and Parkinson's Diseases and in treating conditions associated with such disorders. Such modified peptides have affinity for GLP-1R, GIP-R and/or GCGR, as can be determined for example by a cellular assay as described herein or, using another assay for making such determinations. In some embodiments, the modified peptide is derived from any one of SEQ ID NOS. 1-29; or a derivative thereof, such as a conservatively substituted derivative thereof; a modified peptide comprising a peptide selected from the group consisting of GGG Tri-Agonist, GIP/GLP Coagonist Peptide, GIP/GLP Coagonist Peptide II, C2816, a GLP-1/cholecystokinin receptor-1 (CCK1) co-agonist, ZP3022, a GLP-1/gastrin co-agonist, GLP-1/xenin co-agonist, GIP/xenin co-agonist, GLP-1/gastrin/xenin tri-agonist, NNC 9204-1177 (NN9277), LY3305677, JNJ-54728518, LY2944876/TT-401, CPD86, LY3298176 (Tirzepatide), LY3437943, SAR438335, ZP-I-98, ZP-DI-70, HM15211, NN9423/MAR423, PB-719, and DD01; and/or combinations thereof; the peptide being modified to comprise at least one surfactant (e.g., in preferred embodiments surfactant X). In some embodiments, the modified peptide exhibits about equal affinity for GLP-1R. GIP-R and/or GCGR as can be determined using the aforementioned cellular assay. In some embodiments, this disclosure provides pharmaceutical dosage formulation of such modified peptide(s) configured to control blood glucose with reduction of one or more adverse events as compared to an unmodified peptide. In some embodiments, this disclosure provides pharmaceutical dosage formulation of such modified peptide(s) configured to induce weight loss with reduction of one or more adverse events as compared the unmodified version of the peptide and/or the parent peptide (e.g., SEQ ID NOS: 1-29). The adverse events being in some embodiments selected from nausea, vomiting, diarrhea, abdominal pain and constipation, upon administration to a mammal. Those adverse events are typically observed following administration of a (dual) agonist with rapid entry into the circulation, leading to an excessively high Cmax. In some embodiments, administration of the modified peptide(s) can result in improvements in other results (e.g., weight loss, chronic weight management, fat loss, lipid profile) and/or pharmacokinetic (PK) parameters as compared to an unmodified peptide (e.g., semaglutide). Other aspects of this disclosure are also contemplated as will be understood from the same by those of ordinary skill in the art.

### DETAILED DESCRIPTION OF THE DISCLOSURE

This disclosure relates to agonist peptide product(s) as well as pharmaceutical dosage formulations comprising, and methods for using, the same. The agonist peptides have affinity for, glucagon-like peptide 1 receptor (GLP-1R), gastric inhibitory polypeptide receptor (GIP-R) and/or glucagon receptor (GCGR), as may be determined using a cellular assay, wherein the peptides are conjugated to a non-ionic glycolipid surfactant. The peptides may be selective for one receptor, or in certain embodiments have dual or tri-binding affinity properties for the respective receptors (e.g. GLP-1R, GIP-R and/or GCGR). In some embodiments, this disclosure provides pharmaceutical dosage formulations configured to control blood glucose or induce weight loss. In certain embodiments, and without being bound by theory, it is believed that conjugation of the non-ionic glycolipid surfactant (as defined below wherein the surfactant X is attached to an amino acid U of the parent peptide) in the peptide products improves tolerability (as compared to other surfactants such as PEG). In certain embodiment, amino-acid residue at position 2 in the parent peptide is substitute by D-Serine or 2-Aminoisobutyric acid. In certain embodiment, residues at position 16 and 20 in the parent peptide may be substituted by a Glutamic acid and Lysine respectively to form a lactam bridge. In other embodiments, amino-acid residue 17 or amino acid residue 20 in the parent peptide may be substituted by a Lysine residue to be employed for the conjugate with surfactant X. The peptide products comprising the peptide and non-ionic glycolipid surfactant form micelles, especially in a stable formulation configured for critical micelle concentration for each peptide product, which may at the site of subcutaneous administration create a durable depot effect causing slower release (i.e., slower protraction) of the peptide product into the blood (e.g., as seen with a low Cmax).

In some embodiments, such pharmaceutical dosage formulations exhibit a reduction in adverse events as compared to an agonist that does not comprise a present non-ionic glycolipid surfactant (as represented below by Formula I). In some embodiments, the adverse events can include nausea, vomiting, diarrhea, abdominal pain and/or constipation, that are typically observed following administration of an agonist without a present surfactant to a mammal. In some embodiments, this disclosure provides novel peptide-based glucagon-like peptide 1 receptor (GLP-1R), gastric inhibitory polypeptide receptor (GIP-R) and/or glucagon receptor (GCGR) agonist peptide products designed to treat the underlying metabolic dysfunction that leads to non-alcoholic steatohepatitis (NASH). In other embodiments, this disclosure provides novel peptide-based glucagon-like peptide 1 receptor (GLP-1R), gastric inhibitory polypeptide receptor (GIPR) and/or glucagon receptor (GCGR) agonist peptide products designed to treat the underlying metabolic dysfunction that leads to obesity.

In some embodiments, the modified peptide (a peptide modified to comprise at least one non-ionic glycolipid surfactant such as X herein) is derived from any one of the peptides of SEQ ID NOS: 1-29 shown below, or a derivative thereof:
HAQGTFTSDYSKYLDE*KRAK*EFVQWLMNT-Z (SEQ ID NO: 1; sequence derived from HM12525A/JNJ-64565111 (Efinopegdutide), Hanmi Pharmaceuticals, GLP-1R/GCGR);
Z-HSQGTFTSDYSKYLDSRRAQDFVQWLMNTKRNRNNIA (SEQ ID NO: 2; sequence derived from MOD6030/1; Prolor/OPKO Biological; GLP-1R/GCGR);
HS^{d}QGTFTSDLSKQKESKAAQDFIEWLKAGGPSSGAPPPS-NH₂ (SEQ ID NO: 3; sequence derived from SAR425899; Sanofi; GLP-1R/GCGR);
HSQGTFTSDKSEYLDSERARDFVAWLEAGG (SEQ ID NO: 4; sequence derived from MEDI0382; MedImmune; GLP-1R/GCGR);
HAibQGTFTSDKSKYLDERAAQDFVQWLLDT (SEQ ID NO: 5; sequence derived from MK8521; Merck; GLP-1R/GCGR);
HAibQGTFTSDYSKYLDERAAKDFIEWLEKGSGSGGA (SEQ ID NO: 6; sequence derived from ZP2929/BI 456906; Zealand-Boehringer; GLP-1R/GCGR);
YAibQGTFTSDYSIYLDKQAAAibQFVNYLLAGGPSSGAPPPSK (SEQ ID NO: 7; sequence derived from NN9709/NNC0090-2746/MAR709/RG7697/R06811135; Novo Nordisk; GLP-1R/GIPR);
YX1EGTFTSDYSIX2LDKIAQK*AX3VQWLIAGGPSSGAPPPS (SEQ ID NO: 8) or Y(Aib)GTFTSDYSI(Aib)LDKIAQK*A(F or 1-Nal)VQWLIAGGPSSGAPPPS (SEQ ID NO: 9; as described in U.S. Pat. No. 9,474,780 and WO 2016/111971; GIP-GLP-1 co-agonist) wherein X1 is Aib; X2 is Aib; K* at position 20 is chemically modified through conjugation to the epsilon-amino group of the K side-chain with ([2-(2-Amino-ethoxy)-ethoxy]-acetyl) 2-(YGlu)a-CO—(CH₂)b-CO2H wherein a is 1 to 2 and b is 10 to 20; X3 is Phe or 1-Nal; and the C-terminal amino acid is optionally amidated as a C-terminal primary amide (as in SEQ ID NO: 12), or a pharmaceutically acceptable salt thereof;
YSHGTFTSDVSKYLD-Aib-KKAAEFVAWLLEE (SEQ ID NO: 10; described in US 9688736 and WO 2015/094878);

HAQGTFLSDYSKYLD-Aib-KKAQEFVEWLLKTGPSSGAPPKSK (SEQ ID NO: 11; described in US 9,695,225 and WO 2015/094875);

YSQGTFTSDYSKYLD-Aib-KKAQEFVEWLLKTGPSSGAPPKSK (SEQ ID NO: 12; as described in US 2016/0311882 and WO 2015/094876);

H-Aib-QGTFTSDYSKYLDEKKAKEFVEWLLEGGPSSG (SEQ ID NO: 13; as described in US 9,938,335 and WO 2016/209707);

H-Aib-QGTFTSDYSKYLDEKKAKEFVEWLLSGGPSSG (SEQ ID NO: 14; as described in US 9,938,335 and WO 2016/209707);

YX2X3GTX6TSDYSIX13LDKX17AQX20AFIEYLLEGGPSSGAPPPS (SEQ ID NO: 15, as described in WO 2019/125929), where X2 is Aib, X3 is Q or H, X6 is aMeF or aMeF(2F), X13 is L or aMeL, X17 is any amino acid with a functional group available for conjugation, where the functional group is conjugated to a C16-C22 fatty acid, X20 is Aib, Q or H, and the C-terminal amino acid is optionally amidated;

R1X1X2X3GTX6TSDX10X11X12X13X14DX16X17AX19X20X21X22X23X24X25X26X 27X28X29X30X31, wherein X is any amino acid (SEQ ID NO: 16; as described in US 2020/0024322 or WO 2020/023386);
HSQGTFTSDYSKYLDSRRAQDFVQWLMNT (SEQ ID NO: 17; glucagon); and,
YAEGTFISDYSIAMDKIHQQDFVNWLLAQKGKKNDWKHNITQ (SEQ ID NO: 18; gastric inhibitory polypeptide (GIP), or glucose-dependent insulinotropic polypeptide);
HAEGTFTSDVSSYLEGQAAKEFIAWLVKGRG (SEQ ID NO: 19; GLP-1 (7-37));
HAEGTFTSDVSSYLEGQAAKEFIAWLVRGRG (SEQ ID NO: 20; Liraglutide), wherein in certain embodiments the underlined residue is conjugation site for fatty acid modification (e.g. non-ionic glycolipid surfactant);
HXEGTFTSDVSSYLEGQAAKEFIAWLVRGRG where X is Aib (SEQ ID NO: 21; Semaglutide), wherein in certain embodiments underlined residue is a conjugation site for fatty acid modification (e.g. non-ionic glycolipid surfactant);
HGEGTFTSDVSSYLEEQAAKEFIAWLVKGGGZ (SEQ ID NO: 22; sequence derived from Dilaglutide), wherein in certain embodiments the present non-ionic glycolipid surfactant can be conjugated via terminal lysine residue in place of Z (e.g. addition of a Z is a C-terminus conjugation site);
HSQGTFTSDYSKYLDSRRAQDFVQWLMNT-OH (SEQ ID NO: 23; Glucagen), wherein in certain embodiments underlined residue is a conjugation site for fatty acid modification (e.g. non-ionic glycolipid surfactant);
HGDGSFSDEMNTILDNLAARDFINWLIQTKITD (SEQ ID NO: 24; Teduglutide), wherein in certain embodiments the underlined residue is a conjugation site for fatty acid modification (e.g. non-ionic glycolipid surfactant);
Y-Aib-EGTFTSDYSI-Aib-LDKIAQKAFVQWLIAGGPSSGAPPPS (SEQ ID NO:25; Tirzepatide) wherein in certain embodiments the underlined residue is a conjugation site for fatty acid modification (e.g. non-ionic glycolipid surfactant);
H-Aib-HGTFTSDLSKLKEEQRQ-Aib-EFIEWLKA-dAla-GPPS-Aib-KPPPK (SEQ ID NO:26; SAR441255) wherein in certain embodiments the underlined residue is a conjugation site for fatty acid modification (e.g. non-ionic glycolipid surfactant);
YX2QGTFTSDYSIX13LDKKAX19X20AFIEYLLX28X29GPSSX34APPPS (SEQ ID NO:27 as described in WO2019125938A1) , wherein: X2 is Aib, X13 is L or aMeL, X19 is Q or A, X20 is Aib, αMeK, Q or H, X28 is E or A, X29 is G or Aib, X34 is G or Aib and wherein in certain embodiments the underlined residue is a conjugation site for fatty acid modification (e.g. non-ionic glycolipid surfactant);
Xaa1-Xaa2-Xaa3-Gly-Thr-Phe-Xaa7-Ser-Asp-Xaa10-Ser-Xaa12-Xaa13-Xaa14-Xaa15-Xaa 16-Xaa17-Xaa18-Xaa19-Xaa20-Xaa21-Phe-Xaa23-Xaa24-Trp-Leu-Xaa27-Xaa28-Xaa 29-Xaa30 (SEQ ID NO: 28 as described in US2019153060A1) wherein in certain embodiments the underlined residue is a conjugation site for fatty acid modification (e.g. non-ionic glycolipid surfactant);. and,
HSQGTFTSDKSEYLDSERARDFVAWLEAGG (SEQ ID NO: 29; Cotadutide) wherein in certain embodiments the underlined residue is a conjugation site for fatty acid modification (e.g. non-ionic glycolipid surfactant);
wherein the underlined residues indicate a conjugation site, or wherein Z indicates N- or C-terminus conjugation site, and/or a conjugation site may be U as described below (i.e., an underlined residue or Z can be U); Aib indicates 2-Aminoisobutyric acid; dAla indicates D-alanine, 1-Nal indicates 1-Naphthyl)-L-alanine, Sd indicates D-Serine; αMeK indicates methylated lysine residues; * indicates residues engaged in lactam bridge; and, underlined residues correspond to conjugation sites.

A sequence corresponding to SEQ ID NO: 1 to SEQ ID NO:29 to wherein the C-terminal amino acid is optionally amidated. A sequence corresponding to SEQ ID NO: 1 to SEQ ID NO:29 to wherein lysine residues on position 10, 14, 16, 17 or 20 can alternatively be used as a conjugation site for fatty acid modification (e.g. non-ionic glycolipid surfactant).

Other peptides suitable for use as disclosed herein can include GGG Tri-Agonist (Eli Lilly); GIP/GLP Coagonist Peptide (Eli Lilly); GIP/GLP Coagonist Peptide II (Eli Lilly); C2816 (Medimmune), a GLP-1/cholecystokinin receptor-1 (CCK1) co-agonist; ZP3022 (Zealand), a GLP-1/gastrin co-agonist; GLP-1/xenin co-agonist (University of Ulster); GIP/xenin co-agonist (University of Ulster); and, GLP-1/gastrin/xenin tri-agonist (University of Ulster); NNC 9204-1177 (NN9277) (Novo Nordisk; GLP-1R/GCGR); LY3305677 (Eli Lilly; GLP-1R/GCGR); JNJ-54728518 (Janssen Pharmaceuticals; GLP-1R/GCGR); LY2944876/TT-401 (Transition Therapeutics; GLP-1R/GCGR); CPD86 (Eli Lilly; GLP-1R/GIPR); LY3298176 (Tirzepatide); (Eli Lilly; GLP-1R/GIPR); LY3437943 (Eli Lilly; GLP-1R/GCGR/GIPR); SAR438335 (Sanofi; GLP-1R/GIPR); ZP-I-98 (Zealand; GLP-1R/GIPR); ZP-DI-70 (Zealand; GLP-1R/GIPR); HM15211 (Hanmi Pharmaceuticals; GLP-1R/GIPR/GCGR); NN9423/MAR423 (Novo Nordisk/Marcadia; GLP-1R/GIPR/GCGR), PB-719 (PegBio, GLP-1R/GIPR); and, DD01 (D&D Pharma, GLP-1R/GIPR). The amino acid sequences of any of the peptides disclosed above can be substituted as desired so long as activity of those peptides is maintained and/or that activity is not significantly diminished.

The modified peptides of this disclosure include at least one amino acid “U”, which is a natural or unnatural amino acid comprising a functional group used for covalent attachment to a surfactant X (preferably a hydrophobic surfactant; e.g., as described in U.S. Pat. No. 9,856,306), where U may be an amino acid present in the peptide or added to the peptide by substitution or addition, such peptides being referred to herein as “modified peptides”. In some embodiments, surfactant X can be covalently attached to a peptide, the peptide comprising a linker amino acid U and at least one other amino acid as in Formula I-A:

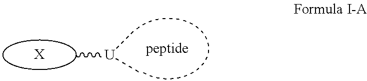

wherein the surfactant X is a group of Formula I:

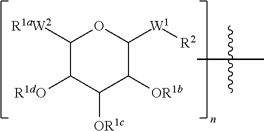

R^{a} is independently, at each occurrence, a bond, H, a protecting group, a substituted or unsubstituted C₁-C30 alkyl group, a saccharide, a substituted or unsubstituted alkoxyaryl group, or a substituted or unsubstituted aralkyl group;

R^{1b}, Pv^{1c}, and R^{1d} are each, independently at each occurrence, a bond, H, a protecting group, a substituted or unsubstituted C₁-C₃₀ alkyl group, a substituted or unsubstituted alkoxyaryl group, or a substituted or unsubstituted aralkyl group;

W¹ is independently, at each occurrence, —CH₂—, —CH₂—O—, —(C═O), —(C═O)—O—, (C═O)—NH—, —(C═S)—, —(C═S)—NH—, or —CH₂—S—;

W² is —O—, —CH₂— or —S—;

R is independently, at each occurrence, a bond to U, H, a substituted or unsubstituted C₁-C₃₀ alkyl group, a substituted or unsubstituted alkoxyaryl group, or a substituted or unsubstituted aralkyl group, —NH, —S—, -triazolo-, —NH(C═O)—CH₂—, —(CH₂)ₘ-maleimide-;

n is 1, 2 or 3; and,

m is an integer of 1-10.

In some embodiments, n is 1. In some embodiments, n is 2, and a first glycoside is attached to a second glycoside via bond between W² of the first glycoside and any one of OR^{1b}, OR^{1c} or OR^{1d} of the second glycoside. In some embodiments, n is 3, and a first glycoside is attached to a second glycoside via bond between W² of the first glycoside and any one of OR^{1b}, OR^{1c} or OR^{1d} of the second glycoside, and the second glycoside is attached to a third glycoside via bond between W² of the second glycoside and any one of OR^{1b}, OR^{1c} or OR^{1d} of the third glycoside.

In one embodiment, compounds of Formula I-A are compounds wherein X has the structure:

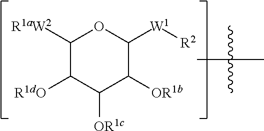

wherein:

R^{1a} is H, a protecting group, a saccharide, a substituted or unsubstituted C₁-C30 alkyl group, or a steroid nucleus containing moiety;

R^{1b}, R^{1c}, and R^{1d} are each, independently at each occurrence, H, a protecting group, or a substituted or unsubstituted C₁-C30 alkyl group;

W¹ is independently, at each occurrence, —CH₂—, —CH₂—O—, —(C═O), —(C═O)—O—, (C═O)—NH—, —(C═S)—, —(C═S)—NH—, or —CH₂—S—;

W² is —O—, —S—;

R² is a bond to U, —NH—, —S—, —NH(C═O)—CH₂— or —(CH₂)ₘ-maleimide-, and,

m is 1-10.

In another embodiment, compounds of Formula I-A are compounds wherein X has the structure:

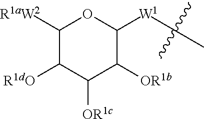

Accordingly, in the embodiment described above, R is a bond.

For instance, in an exemplary embodiment of the structure of X described above, W¹ is —C(═O)NH—, R″ is a bond between W¹ and an amino acid residue U within the peptide (e.g., an amino group in the sidechain of a lysine residue present in the peptide).

In a further embodiment, compounds of Formula I-A are compounds wherein X has the structure:

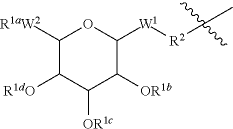

For instance, in an exemplary embodiment of the structure of X described above, W is —CH₂— and R is an alkyl-linked maleimide functional group on X and R is attached to a suitable moiety of an amino acid residue U within the peptide (e.g., a thiol group in a cysteine residue of the peptide forms a thioether with the maleimide on X).

In yet another embodiment, compounds of Formula I-A are compounds wherein X has the structure:

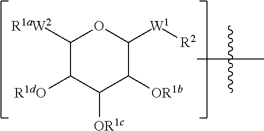

wherein:

R^{a} is H, a protecting group, a saccharide, a substituted or unsubstituted C1-C30 alkyl group, or a steroid nucleus containing moiety;

R^{1b}, R^{1c}, and R^{1d} are each, independently at each occurrence, H, a protecting group, or a substituted or unsubstituted C1-C30 alkyl group;

W¹ is —(C═O)—NH—;

W² is —O—; and,

R is a bond.

In an additional embodiment, compounds of Formula I-A are compounds wherein X has the structure:

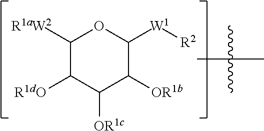

wherein:

R^{a} is a substituted or unsubstituted C₁-C₃₀ alkyl group;

R^{1b}, R^{1c}, and R^{1d} are H;

W¹ is —(C═O)—NH—;

W² is —O—; and

R is a bond.

In some embodiments described above and herein, R^{1a} is a substituted or unsubstituted C₁-C₃₀ alkyl group.

In some embodiments described above and herein, R^{a} is a substituted or unsubstituted C₆-C₂₀ alkyl group.

In some embodiments described above and herein, R^{1a} is a saccharide. In some embodiments, the saccharide is a galactose. In certain embodiments, the saccharide is an alpha-linked galactose. In other embodiments, the saccharide is alpha-linked galactopyranose, beta-linked galactopyranose, alpha-linked galactofuranose, or beta-linked galactofuranose.

Also contemplated herein are alternate embodiments wherein X in Formula I-A has the structure:

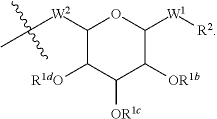

For instance, in an exemplary embodiment of the structure of X described above, W¹ is —S—, R² is a C₁-C₃₀ alkyl group, W² is S, R^{1a} is a bond between W² and a suitable moiety of an amino acid residue U within the peptide (e.g., a thiol group in a cysteine residue of the peptide forms a thioether with X).

In another exemplary embodiment of the structure of X described above, W is —O—, R is a C₁-C₃₀ alkyl group, W² is O, R^{1a} is a bond between W² and a suitable moiety of an amino acid residue U within the peptide (e.g., a hydroxyl group in a serine or threonine residue of the peptide forms an ether with X).

In another exemplary embodiment of the structure of X described above, W is —O—, R is a C₁-C₃₀ alkyl group, W is CO, R is a spacer amino acid structure such as Glum or Lysm that links to a suitable moiety of an amino acid residue U within the peptide (for example, a Glu spacer linked through its gamma CO to the epsilon amino function of a Lys in the peptide or a Lys linked through its alpha CO to a the epsilon amino function of a Lys in the peptide).

In some embodiments, U is used for covalent attachment to X and is a dibasic natural or unnatural amino acid, a natural or unnatural amino acid comprising a thiol, an unnatural amino acid comprising a —N₃ group, an unnatural amino acid comprising an acetylenic group, or an unnatural amino acid comprising a —NH—C(═O)—CH₂—Br or a —(CH₂)ₘ-maleimide, wherein m is 1-10.

In some embodiments of the modified peptides, the surfactant X is an 1-alkyl glycoside class surfactant. In some embodiments of the peptide product, the surfactant is attached to the peptide via an amide bond.

In some embodiments of the peptide product, the surfactant X comprises 1-eicosyl beta-D-glucuronic acid, 1-octadecyl beta-D-glucuronic acid, 1-hexadecyl beta-D-glucuronic acid, 1-tetradecyl beta-D-glucuronic acid, 1-dodecyl beta D-glucuronic acid, 1-decyl beta-D-glucuronic acid, 1-octyl beta-D-glucuronic acid, 1-eicosyl beta-D-diglucuronic acid, 1-octadecyl beta-D-diglucuronic acid, 1-hexadecyl beta-D-diglucuronic acid, 1-tetradecyl beta-D-diglucuronic acid, 1-dodecyl beta-D-diglucuronic acid, 1-decyl beta-D-diglucuronic acid, 1-octyl beta-D-diglucuronic acid, or functionalized 1-eicosyl beta-D-glucose, 1-octadecyl beta-D-glucose, 1-hexadecyl beta-D-glucose, 1-tetradecyl beta-D-glucose, 1-dodecyl beta-D-glucose, 1-decyl beta-D-glucose, 1-octyl beta-D-glucose, 1-eicosyl beta-D-maltoside, 1-octadecyl beta-D-maltoside, 1-hexadecyl beta-D-maltoside, 1-tetradecyl beta-D-maltoside, 1-dodecyl beta-D-maltoside, 1-decyl beta-D-maltoside, 1-octyl beta-D-maltoside, 1-eicosyl beta-D-melibioside, 1-octadecyl beta-D-melibioside, 1-hexadecyl beta-D-melibioside, 1-tetradecyl beta-D-melibioside, 1-dodecyl beta-D-melibioside, 1-decyl beta-D-melibioside, 1-octyl beta-D-melibioside and the like, as well as the corresponding 6′ or 6′, 6 carboxylic acids and the peptide product is prepared by formation of a linkage between the aforementioned groups and a group on the peptide (e.g., a —COOH group in the aforementioned groups and an amino group of the peptide). In some embodiments, the surfactant X is 1-tetradecyl beta-D-maltoside, 1-dodecyl beta-D-maltoside, 1-decyl beta-D-maltoside, 1-octyl beta-D-maltoside, 1-eicosyl beta-D-melibioside, 1-octadecyl beta-D-melibioside, 1-hexadecyl beta-D-melibioside, 1-tetradecyl beta-D-melibioside, 1-dodecyl beta-D-melibioside, 1-decyl beta-D-melibioside, or 1-octyl beta-D-melibioside, as well as the corresponding 6′ or 6′, 6 carboxylic acids. In some embodiments, the surfactant X is 1-tetradecyl beta-D-maltoside, 1-eicosyl beta-D-melibioside, 1-octadecyl beta-D-melibioside, 1-hexadecyl beta-D-melibioside, 1-tetradecyl beta-D-melibioside, 1-dodecyl beta-D-melibioside, 1-decyl beta-D-melibioside, or 1-octyl beta-D-melibioside.

In some embodiments of the peptide product, U is a terminal amino acid of the peptide. In some embodiments of the peptide product, U is a non-terminal amino acid of the peptide. In some embodiments of the peptide product, U is a natural D- or L-amino acid. In some embodiments of the peptide product, U is an unnatural amino acid. In some embodiments of the peptide product, U is selected from Lys, Cys, Orn, or an unnatural amino acid comprising a functional group used for covalent attachment to the surfactant X.

In some embodiments of the peptide product, the functional group used for covalent attachment of the peptide to the surfactant X is —NH₂, —SH, —OH, —N₃, haloacetyl, a —(CH₂)ₘ-maleimide (wherein m is 1-10), or an acetylenic group.

In some embodiments side chain functional groups of two different amino acid residues are linked to form a cyclic lactam. This linkage is denoted with an asterisk on the two residues so linked. For example, in some embodiments, a Lys* side chain forms a cyclic lactam with the side chain of Glu*. In some embodiments such lactam structures are reversed and are formed from a Glu* and a Lys*. Such lactam linkages in some instances are known to stabilize alpha helical structures in peptides (Condon, S. M., et al. (2002) Bioorg Med Chem 10: 731-736; Murage, E. N., et al (2008) Bioorg Med Chem 16: 10106-12); Murage, E. N., et al. (2010) J Med Chem 53: 6412-20). In some embodiments cysteine residues may be linked through disulfide formation in order to accomplish a similar form of conformational restriction and assist in the formation of helical structures (Li, Y., et al. (2011) Peptides 32: 1400-1407). In some embodiments side chain functional groups of two different amino acid residues are linked to form a heterocycle generated through a “click reaction” between side chain azide and alkyne functional groups in order to achieve a similar form of conformational restriction and stabilized helical conformations (Le Chevalier Isaad A., et al. (2009) J Peptide Sci 15: 451 -4). In some embodiments side chain functional groups of two different amino acid residues are linked to form a C—C double bond through the use of an olefin metathesis reaction and may be further modified by reduction to a C—C single bond (Verdine, G. L. and Hilinski, G. J. (2011) Meth Enzymol 503: 3-33).

In some embodiments, the peptide product comprising a covalently linked alkyl glycoside is a covalently modified glucagon or analog thereof In some of such embodiments, the peptide product contains a covalently linked 1 —O-alkyl β-D-glucuronic acid and the peptide is an analog of glucagon.

In some embodiments, a peptide product comprising a covalently linked alkyl glycoside is a covalently modified GLP-1, or analog thereof. In some of such embodiments, the peptide product comprises a covalently linked 1 —O-alkyl β-D-glucuronic acid and the peptide is an analog of GLP-1.

In some embodiments, a peptide product comprising a covalently linked alkyl glycoside is a covalently modified gastric inhibitory polypeptide (GIP), or analog thereof. In some of such embodiments, the peptide product comprises a covalently linked 1 —O-alkyl β-D-glucuronic acid and the peptide is an analog of gastric inhibitory polypeptide.

In preferred embodiments, the modified peptide is one having the amino acid sequence of any one of the peptides of SEQ ID NOS: 1-29, or a derivative or variant thereof.

In some embodiments, the modified peptide is formulated as a solution for injection comprising pharmaceutically acceptable excipients such as a osmolarity adjusting agent or salt, a buffering agent, an stabilizing agent and/or a surfactant, a pH adjuster, a preservative and a solvent. In some embodiment, the osmolarity adjusting agent is mannitol, sorbitol, glycerol, and glycine, propylene glycol or sodium chloride. In some embodiments, the buffering agent is histidine arginine, lysine, phosphate, acetate, carbonate, bicarbonate, citrate, Meglumine or Tris. In some embodiments, the stabilizing agent is histidine, arginine or lysine. In some embodiments, the surfactant is polysorbate 20 or polysorbate 80. In some embodiment, the pH adjuster is hydrochloric acid and/or sodium hydroxide. In some embodiments, The presevrative is selected from Methyl Paraben, Ethyl Paraben, Propyl Paraben, Butyl Paraben, Benzyl Alcohol, Chlorobutanol, Phenol, Meta cresol, Chloro cresol, Benzoic acid, Sorbic acid, Thiomersal, Phenylmercuric nitrate, Bronopol, Propylene Glycol, Benzylkonium Chloride, Benzethonium Chloride. In preferred embodiment, the osmolarity adjusting agent is mannitol, the buffering agent and stabilizing agent is arginine, and the surfactant is a polysorbate 20. In some embodiments, the dual agonist peptide can be formulated as a pharmaceutical dosage formulation comprising about 0.025-0.15% (w/w) polysorbate 20, about 0.2-0.5% (w/w) arginine, and about 3-6% (w/w) mannitol in deionized water. In some embodiments, the pharmaceutical dosage formulation comprises any one of the peptides of SEQ ID NOS: 1-29, or a derivative or variant thereof in a formulation comprising, consisting essentially of, or consisting of, about 0.050% (w/w) polysorbate 20, about 0.35% (w/w) arginine, and about 4.3% (w/w) mannitol in water. In some embodiments, the pharmaceutical dosage formulation any one of the peptides of SEQ ID NOS: 1-29, or a derivative or variant thereof at a concentration ranging from 0.05 mg/ml to 20 mg/ml, preferably from 0.1 mg/ml to 10 mg/ml or more preferably 0.5 mg/mg to 10 mg/ml. In some embodiments, the pH of the pharmaceutical dosage formulation comprising any one of the peptides of SEQ ID NOS: 1-29, or a derivative or variant thereof is from 6 to 10, more preferably 6 to 8.

The modified peptides (e.g., SEQ ID NOS: 1-29, and/or derivatives or variants thereof) is obtained by chemical synthesis such as solid-phase peptide synthesis (SPPS). As described in Behrendt R, White P, Offer J. Advances in Fmoc solid-phase peptide synthesis. J Pept Sci. 2016 Jan;22(1):4-27. In some embodiments, the modified peptides can include one or more conservatively substituted amino acids as described herein.

In some embodiments, the activity of a modified peptide can be determined by a cellular assay such as that described in Example 2 herein. Briefly, in some embodiments, cellular assays can be carried out by measuring cAMP stimulation or arrestin activation in CHO cells into which human GLP-1R GIP-R, or GCGR are expressed (LeadFlunter assays (DiscoveRx). Preferably, such assays are carried out in the presence of 0.1% ovalbumin as compared to 0.1% bovine serum albumin (BSA) as may be typical, since the modified peptides of SEQ ID NOS: 1-29 can bind very tightly to serum albumin (>99%) and distort the results (see, e.g., Example 2 herein). In some embodiments, as determined using such assays, the modified peptide can have affinity for GLP-1R, GIP-R and/or GCGR.

A “peptide” (e.g., modified peptide) comprises two or more natural or/and unnatural amino acid residues linked typically via peptide bonds. Such amino acids can include naturally occurring structural variants, naturally occurring non-proteinogenic amino acids, or/and synthetic non-naturally occurring analogs of natural amino acids. The terms “peptide” and “polypeptide” are used interchangeably herein. Peptides include short peptides (about 2-20 amino acids), medium-length peptides (about 21-50 amino acids) and long peptides (>about 50 amino acids, which can also be called “proteins”). In some embodiments, a peptide product comprises a surfactant moiety covalently and stably attached to a peptide of no more than about 50, 40 or 30 amino acids. Synthetic peptides can be synthesized using an automated peptide synthesizer, for example. Peptides can also be produced recombinantly in cells expressing nucleic acid sequences that encode the peptides. Conventional notation is used herein to portray peptide sequences: the left-hand end of a peptide sequence is the amino (N)-terminus, and the right-hand end of a peptide sequence is the carboxyl (C)-terminus. Standard one-letter and three-letter abbreviations for the common amino acids are used herein. Although the abbreviations used in the amino acid sequences disclosed herein represent L-amino acids unless otherwise designated as D- or DL- or the amino acid is achiral, the counterpart D-isomer generally can be used at any position (e.g., to resist proteolytic degradation). Abbreviations for other amino acids used herein include: Aib=a-aminoisobutyric acid (or 2-methylalanine or Ca-methylalanine); Xaa: any amino acid, typically specifically defined within a formula. Abbreviations for other amino acids that can be used as described herein include: Ac3c=1-aminocyclopropane-1-carboxylic acid; Ac4c=1-aminocyclobutane-1-carboxylic acid; Ac5c=1-aminocyclopentane-1-carboxylic acid; Ac6c=1-aminocyclohexane-1-carboxylic acid; Aib=alpha-aminoisobutyric acid (or 2-methylalanine or Calpha-methylalanine); Bip=3-(biphenyl-4-yl)alanine; Bip2Et=3-(2′-ethylbiphenyl-4-yl)alanine; Bip2EtMeO=3-(2′-ethyl-4′-methoxybiphenyl-4-yl)alanine; Cit=citrulline; Deg=2,2-diethylglycine; Dmt=(2,6-dimethyl)tyrosine; 2FPhe=(2-fluorophenyl)alanine; 2FMePhe or 2FaMePhe=Ca-methyl-(2-fluorophenyl)alanine; hArg=homoarginine; MeLys or aMeLys=Ca-methyllysine; MePhe or aMePhe=Ca-methylphenylalanine; MePro or aMePro=Ca-methylproline; Nal1 or Nal(1)=3-(1-naphthypalanine; Nal2 or Nal(2)=3-(2-naphthyl)alanine; Nle=norleucine; Om=ornithine; and Tmp=(2,4,6-trimethylphenyl)alanine; 1,2,3,4-tetrahydroisoquinoline-3-carboxylic acid (Tic) and a Tic-Phe dipeptide moiety with a reduced amide bond between the residues (designated as Tic-Ψ[CFl2-NFl]-Ψ-Phe) have the following structures:

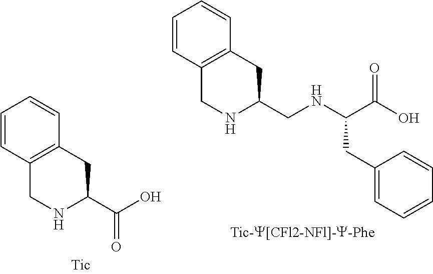 [SMILES: O=C(O)[C@@H]1Cc2ccccc2CN1 ; O=C(O)[C@H](Cc1ccccc1)NC[C@@H]1Cc2ccccc2CN1]

Unless specifically stated otherwise or the context clearly indicates otherwise, the disclosure encompasses any and all forms of a modified peptide that may be produced, whether the modified peptide is produced synthetically (e.g., using a peptide synthesizer) or by a cell (e.g., by recombinant production). Such forms of a modified peptide can include one or more modifications that may be made during the course of synthetic or cellular production of the peptide, such as one or more post-translational modifications, whether or not the one or more modifications are deliberate. A modified peptide can have the same type of modification at two or more different places, or/and can have two or more different types of modifications. Modifications that may be made during the course of synthetic or cellular production of a modified peptide, including chemical and post-translational modifications, include without limitation glycosylation (e.g., N-linked glycosylation and O-linked glycosylation), lipidation, phosphorylation, sulfation, acetylation (e.g., acetylation of the N-terminus), amidation (e.g., amidation of the C-terminus), hydroxylation, methylation, formation of an intramolecular or intermolecular disulfide bond, formation of a lactam between two side chains, formation of pyroglutamate, and ubiquitination. A modified peptide can have one or more modifications anywhere, such as the N-terminus, the C-terminus, one or more amino acid side chains, or the modified peptide backbone, or any combination thereof. In some embodiments, a modified peptide is acetylated at the N-terminus or/and has a carboxamide (—CONH₂) group at the C-terminus, which can increase the stability of the modified peptide.

Potential modifications of a modified peptide also include deletion of one or more amino acids, addition/insertion of one or more natural or/and unnatural amino acids, or substitution with one or more natural or/and unnatural amino acids, or any combination or all thereof. A substitution can be conservative or non-conservative. Such modifications may be deliberate, such as via site-directed mutagenesis or in the chemical synthesis of a modified peptide, or may be accidental, such as via mutations arising in the host cell that produces the modified peptide or via errors due to PCR amplification. An unnatural amino acid can have the same chemical structure as the counterpart natural amino acid but have the D stereochemistry, or it can have a different chemical structure and the D or L stereochemistry. Unnatural amino acids can be utilized, e.g., to promote a-helix formation or/and to increase the stability of the modified peptide (e.g., to resist proteolytic degradation). A modified peptide having one or more modifications relative to a reference modified peptide may be called an “analog” or “variant” of the reference modified peptide as appropriate. An “analog” typically retains one or more essential properties (e.g., receptor binding, activation of a receptor or enzyme, inhibition of a receptor or enzyme, or other biological activity) of the reference modified peptide. A “variant” may or may not retain the biological activity of the reference modified peptide, or/and may have a different biological activity. It is preferred that such a variant maintain its ability to act as an agonist of GLP-1R and GCGR, and in more preferred embodiments, has about equal affinity for GLP-1R and GCGR. In some embodiments, an analog or variant of a reference peptide has a different amino acid sequence than the reference modified peptide.

The term “conservative substitution” refers to substitution of an amino acid in a modified peptide with a functionally, structurally or chemically similar natural or unnatural amino acid. In certain embodiments, the following groups each contain natural amino acids that are conservative substitutions for one another: 1) Glycine (Gly/G), Alanine (Ala/A); 2) Isoleucine (Ile/I), Leucine (Leu/L), Methionine (Met/M), Valine (Val/V); 3) Phenylalanine (Phe/F), Tyrosine (Tyr/Y), Tryptophan (Trp/W); 4) Serine (Ser/S), Threonine (Thr/T), Cysteine (Cys/C); 5) Asparagine (Asn/N), Glutamine (Gln/Q); 6) Aspartic acid (Asp/D), Glutamic acid (Glu/E); and, 7) Arginine (Arg/R), Lysine (Lys/K), Histidine (His/H). In further embodiments, the following groups each contain natural amino acids that are conservative substitutions for one another: 1) non-polar: Ala, Val, Leu, Ile, Met, Pro (proline/P), Phe, Trp; 2) hydrophobic: Val, Leu, Ile, Phe, Trp; 3) aliphatic: Ala, Val, Leu, Ile; 4) aromatic: Phe, Tyr, Trp, His; 5) uncharged polar or hydrophilic: Gly, Ala, Pro, Ser, Thr, Cys, Asn, Gln, Tyr; 6) aliphatic hydroxyl- or sulfhydryl-containing: Ser, Thr, Cys; 7) amide-containing: Asn, Gln; 8) acidic: Asp, Glu; 9) basic: Lys, Arg, His; and, 10) small: Gly, Ala, Ser, Cys. In other embodiments, amino acids may be grouped as conservative substitutions as set out below: 1) hydrophobic: Val, Leu, Ile, Met, Phe, Trp; 2) aromatic: Phe, Tyr, Trp, His; 3) neutral hydrophilic: Gly, Ala, Pro, Ser, Thr, Cys, Asn, Gln; 4) acidic: Asp, Glu; 5) basic: Lys, Arg, His; and, 6) residues that influence backbone orientation: Pro.

Examples of unnatural or non-proteinogenic amino acids include without limitation alanine analogs (e.g., α-ethylGly [α-aminobutyric acid or Abu], α-n-propylGly [norvaline or Nva], α-tert-butylGly [Tbg], α-vinyl Gly [Vg or Vlg], α-allylGly [Alg], α-propargylGly [Prg], 3-cyclopropylAla [Cpa] and Aib), leucine analogs (e.g., nor-leucine, Nle), proline analogs (e.g., α-MePro), phenylalanine analogs (e.g., Phe(2-F), Phe(2-Me), Tmp, Bip, Bip(2′-Et-4′-OMe), Nal1, Nal2, Tic, α-MePhe, α-MePhe(2-F) and α-MePhe(2-Me)), tyrosine analogs (e.g., Dmt and α-MeTyr), serine analogs (e.g., homoserine [isothreonine or hSer]), glutamine analogs (e.g., Cit), arginine analogs (e.g., hArg, N,N′-g-dialkyl-hArg), lysine analogs (e.g, homolysine [hLys], Orn and α-MeLys), α, α-disubstituted amino acids (e.g., Aib, α, α-diethylGly [Deg], α-cyclohexylAla [2-Cha], Ac3c, Ac4c, Ac5c and Ac6c), and other unnatural amino acids disclosed in A. Santoprete et al., Pept. Sci., 17:270-280 (2011). α,α-Di-substituted amino acids can provide conformational restraint or/and a-helix stabilization. A reduced amide bond between two residues (as in, e.g., Tic-Ψ[CF12-NF1]-Ψ-Phe) increases protease resistance and may also, e.g., alter receptor binding. The disclosure encompasses all pharmaceutically acceptable salts of modified peptides, including those with a positive net charge, those with a negative net charge, and those with no net charge.

An “alkyl” group refers to an aliphatic hydrocarbon group. An alkyl group can be saturated or unsaturated, and can be straight-chain (linear), branched or cyclic. In some embodiments, an alkyl group is not cyclic. In some embodiments, an alkyl group contains 1-30, 6-30, 6-20 or 8-20 carbon atoms. A “substituted” alkyl group is substituted with one or more substituents. In some embodiments, the one or more substituents are independently selected from halogens, nitro, cyano, oxo, hydroxy, alkoxy, haloalkoxy, aryloxy, thiol, alkylthio, arylthio, alkylsulfoxide, arylsulfoxide, alkylsulfone, aryl sulfone, amino, alkylamino, dialkylamino, arylamino, alkoyl, carboxyl, carboxylate, esters, amides, carbonates, carbamates, ureas, alkyl, haloalkyl, fluoroalkyl, aralkyl, alkyl chains containing an acyl group, heteroalkyl, heteroali-cyclic, aryl, alkoxyaryl, heteroaryl, hydrophobic natural compounds (e.g., steroids), and the like. In some embodiments, an alkyl group as a substituent is linear or branched C₁-C₆ alkyl, which can be called “lower alkyl”. Non-limiting examples of lower alkyl groups include methyl, ethyl, propyl (including n-propyl and isopropyl), butyl (including all isomeric forms, such as n-butyl, isobutyl, sec-butyl and/er/-butyl), pentyl (including all isomeric forms, such as n-pentyl), and hexyl (including all isomeric forms, such as n-hexyl). In some embodiments, an alkyl group is attached to the Na-atom of a residue (e.g., Tyr or Dmt) of a peptide. In certain embodiments, an N-alkyl group is straight or branched C₁-C₁₀ alkyl, or aryl -substituted alkyl such as benzyl, phenylethyl or the like. One or two alkyl groups can be attached to the Na-atom of the N-terminal residue. In some embodiments, an alkyl group is a 1-alkyl group that is attached to the C-1 position of a saccharide (e.g., glucose) via a glycosidic bond (e.g., an O—, S—, N—or C-glycosidic bond). In some embodiments, such a 1 -alkyl group is an unsubstituted or substituted C1-C30, C₆-C30, C₆-C₂₀ or C₈-C₂₀ alkyl group. In some embodiments, an alkyl group (e.g., a 1-alkyl group) is substituted with one or more (e.g., 2 or 3) groups independently selected from aryl, —OH, —OR¹, —SH, —NH₂, —NHR¹, —N(R¹)₂, oxo (═O), —C(═O)R², carboxyl (—CO₂H), carboxylate (—CO₂—), —C(═O)OR¹, —OC(═O)R³, —C(═O)N(R¹)₂, —NR⁴C(═O)R³, —OC(═O)OR⁵, —OC(═O)N(R¹)₂, —NR⁴C(═O)OR⁵, and —NR⁴C(═O)N(R¹)₂, wherein: R¹ at each occurrence independently is hydrogen, alkyl or aryl, or both occurrences of R¹ and the nitrogen atom to which they are connected form a heterocyclyl or heteroaryl ring; R² at each occurrence independently is alkyl, heterocyclyl, aryl or heteroaryl; R³ at each occurrence independently is hydrogen, alkyl, heterocyclyl, aryl or heteroaryl; R⁴ at each occurrence independently is hydrogen or alkyl; and, R⁵ at each occurrence independently is alkyl or aryl. In some embodiments, an alkyl group (e.g., a 1-alkyl group) is internally or/and terminally substituted with a carboxyl/carboxylate group, an aryl group or an —O-aryl group. In certain embodiments, an alkyl group (e.g., a 1-alkyl group) is substituted with a carboxyl or carboxylate group at the distal end of the alkyl group. In further embodiments, an alkyl group (e.g., a 1-alkyl group) is substituted with an aryl group at the distal end of the alkyl group. In other embodiments, an alkyl group (e.g., a 1-alkyl group) is substituted with an —O-aryl group at the distal end of the alkyl group. The terms “halogen”, “halide” and “halo” refer to fluoride, chloride, bromide and iodide. The term “acyl” refers to —C(═O)R, where R is an aliphatic group that can be saturated or unsaturated, and can be linear, branched or cyclic. In certain embodiments, R contains 1-20, 1-10 or 1-6 carbon atoms. An acyl group can optionally be substituted with one or more groups, such as halogens, oxo, hydroxyl, alkoxy, thiol, alkylthio, amino, alkylamino, dialkylamino, cycloalkyl, aryl, acyl, carboxyl, esters, amides, hydrophobic natural compounds (e.g., steroids), and the like. The terms “heterocyclyl” and “heterocyclic” refer to a monocyclic non-aromatic group or a multicyclic group that contains at least one non-aromatic ring, wherein at least one non-aromatic ring contains one or more heteroatoms independently selected from O, N and S. The non-aromatic ring containing one or more heteroatoms may be attached or fused to one or more saturated, partially unsaturated or aromatic rings. In certain embodiments, a heterocyclyl or heterocyclic group has from 3 to 15, or 3 to 12, or 3 to 10, or 3 to 8, or 3 to 6 ring atoms. Heterocyclyl or heterocyclic groups include without limitation aziridinyl, azetidinyl, pyrrolidinyl, piperidinyl, morpholinyl, piperazinyl, azepanyl, azocanyl, oxiranyl, oxetanyl, tetrahydrofuranyl (oxolanyl), tetrahydropyranyl, oxepanyl and oxocanyl. The term “aryl” refers to a monocyclic aromatic hydrocarbon group or a multicyclic group that contains at least one aromatic hydrocarbon ring. In certain embodiments, an aryl group has from 6 to 15, or 6 to 12, or 6 to 10 ring atoms. Aryl groups include without limitation phenyl, naphthalenyl (naphthyl), fluorenyl, azulenyl, anthryl, phenanthryl, biphenyl and terphenyl. The aromatic hydrocarbon ring of an aryl group may be attached or fused to one or more saturated, partially unsaturated or aromatic rings—e.g., dihydronaphthyl, indenyl, indanyl and tetrahydronaphthyl (tetralinyl). An aryl group can optionally be substituted with one or more (e.g., 2 or 3) substituents independently selected from halogens (including —F and —Cl), cyano, nitro, hydroxyl, alkoxy, thiol, alkylthio, alkylsulfoxide, alkylsulfone, amino, alkylamino, dialkylamino, alkyl, haloalkyl (including fluoroalkyl such as trifluoromethyl), acyl, carboxyl, esters, amides, and the like. The term “heteroaryl” refers to a monocyclic aromatic group or a multicyclic group that contains at least one aromatic ring, wherein at least one aromatic ring contains one or more heteroatoms independently selected from O, N and S. The heteroaromatic ring may be attached or fused to one or more saturated, partially unsaturated or aromatic rings that may contain only carbon atoms or that may contain one or more heteroatoms. In certain embodiments, a heteroaryl group has from 5 to 15, or 5 to 12, or 5 to 10 ring atoms. Monocyclic heteroaryl groups include without limitation pyrrolyl, pyrazolyl, pyrazolinyl, imidazolyl, oxazolyl, isoxazolyl, thiazolyl, thiadiazolyl, isothiazolyl, furanyl, thienyl (thiophenyl), oxadiazolyl, triazolyl, tetrazolyl, pyridyl, pyridonyl, pyrazinyl, pyrimidinyl, pyridazinyl, pyridazinonyl and triazinyl. Non-limiting examples of bicyclic heteroaryl groups include indolyl, benzothiazolyl, benzothiadiazolyl, benzoxazolyl, benzisoxazolyl, benzothienyl (benzothiophenyl), quinolinyl, tetrahydroisoquinolinyl, isoquinolinyl, benzimidazolyl, benzotriazolyl, indolizinyl, benzofuranyl, isobenzofuranyl, chromonyl, coumarinyl, cinnolinyl, quinazolinyl, quinoxalinyl, indazolyl, naphthyridinyl, phthalazinyl, quinazolinyl, purinyl, pyrrol opyridinyl, furopyridinyl, thienopyridinyl, dihydroisoindolyl and tetrahydroquinolinyl.

In some embodiments, for instance, the modified peptides can be associated with a saccharide, such as within a pharmaceutically acceptable composition or lyophilizate. Saccharides include monosaccharides, disaccharides and oligosaccharides (e.g., trisaccharides, tetrasaccharides and so on). A reducing saccharide exists in a ring form and an open-chain form in equilibrium, which generally favors the ring form. A functionalized saccharide of a surfactant moiety has a functional group suitable for forming a stable covalent bond with an amino acid of a modified peptide.

The term “pharmaceutically acceptable” refers to a substance (e.g., an active ingredient or an excipient) that is suitable for use in contact with the tissues and organs of a subject without excessive irritation, allergic response, immunogenicity and toxicity, is commensurate with a reasonable benefit/risk ratio, and is effective for its intended use. A “pharmaceutically acceptable” excipient or carrier of a pharmaceutical composition is also compatible with the other ingredients of the composition.

The term “therapeutically effective amount” refers to an amount of a compound that, when administered to a subject, is sufficient to prevent, reduce the risk of developing, delay the onset of, slow the progression of or cause regression of the medical condition being treated, or to alleviate to some extent the medical condition or one or more symptoms or complications of that condition, at least in some fraction of the subjects taking that compound. The term “therapeutically effective amount” also refers to an amount of a compound that is sufficient to elicit the biological or medical response of a cell, tissue, organ or human which is sought by a medical doctor or clinician.

The terms “treat,” “treating” and “treatment” include alleviating, ameliorating, inhibiting the progress of, reversing or abrogating a medical condition or one or more symptoms or complications associated with the condition, and alleviating, ameliorating or eradicating one or more causes of the condition. Reference to “treatment” of a medical condition includes prevention of the condition. The terms “prevent”, “preventing” and “prevention” include precluding, reducing the risk of developing and delaying the onset of a medical condition or one or more symptoms or complications associated with the condition. The term “medical conditions” (or “conditions” for brevity) includes diseases and disorders. The terms “diseases” and “disorders” are used interchangeably herein.

The disclosure also provides pharmaceutical compositions comprising a modified peptide product described herein or a pharmaceutically acceptable salt thereof, and one or more pharmaceutically acceptable carriers or excipients. A pharmaceutical composition contains a therapeutically effective amount of a peptide product or an appropriate fraction thereof. A composition can optionally contain an additional therapeutic agent. In some embodiments, a peptide product is at least about 90%, 95% or 98% pure. Pharmaceutically acceptable excipients and carriers include pharmaceutically acceptable substances, materials and vehicles. Non-limiting examples of types of excipients include liquid and solid fillers, diluents, binders, lubricants, glidants, surfactants, dispersing agents, disintegration agents, emulsifying agents, wetting agents, suspending agents, thickeners, solvents, isotonic agents, buffers, pH adjusters, absorption-delaying agents, stabilizers, antioxidants, preservatives, antimicrobial agents, antibacterial agents, antifungal agents, chelating agents, adjuvants, sweetening agents, flavoring agents, coloring agents, encapsulating materials and coating materials. The use of such excipients in pharmaceutical formulations is known in the art. For example, conventional vehicles and carriers include without limitation oils (e.g., vegetable oils such as olive oil and sesame oil), aqueous solvents (e.g., saline, buffered saline (e.g., phosphate-buffered saline [PBS]) and isotonic solutions (e.g., Ringer's solution)), and organic solvents (e.g., dimethyl sulfoxide and alcohols [e.g., ethanol, glycerol and propylene glycol]). Except insofar as any conventional excipient or carrier is incompatible with a peptide product, the disclosure encompasses the use of conventional excipients and carriers in formulations containing a peptide product. See, e.g., Remington: The Science and Practice of Pharmacy, 21st Ed., Lippincott Williams & Wilkins (Philadelphia, Pennsylvania) (2005); Handbook of Pharmaceutical Excipients, 5th Ed., Rowe et ah, Eds., The Pharmaceutical Press and the American Pharmaceutical Association (2005); Handbook of Pharmaceutical Additives, 3rd Ed., Ash and Ash, Eds., Gower Publishing Co. (2007); and Pharmaceutical Pre-formulation and Formulation, Gibson, Ed., CRC Press (Boca Raton, Florida) (2004).

An appropriate or suitable formulation can depend on various factors, such as the route of administration chosen. Potential routes of administration of a pharmaceutical composition comprising a peptide product include without limitation oral, parenteral (including intradermal, subcutaneous, intramuscular, intravascular, intravenous, intra-arterial, intraperitoneal, intracavitary and topical), and topical (including transdermal, transmucosal, intranasal (e.g., by nasal spray or drop), ocular (e.g., by eye drop), pulmonary (e.g., by oral or nasal inhalation), buccal, sublingual, rectal (e.g., by suppository), and vaginal (e.g., by suppository). In certain embodiments, a present modified peptide product is administered parenterally (e.g., subcutaneously, intravenously or intramuscularly). In other embodiments, a peptide product is administered by oral inhalation or nasal inhalation or insufflation. In some embodiments, the carrier is an aqueous-based carrier, such as in a parenteral (e.g., subcutaneous, intravenous or intramuscular) formulation. In other embodiments, the carrier is a nonaqueous-based carrier. In certain embodiments, the nonaqueous-based carrier is a hydrofluoroalkane (HFA) or HFA-like solvent that may comprise sub-micron anhydrous a-lactose or/and other excipients, such as in a formulation for administration by oral inhalation or nasal inhalation or insufflation.

In some embodiments, a peptide product is administered parenterally (e.g., subcutaneously, intravenously or intramuscularly) by injection. Parenteral administration bypasses the strongly acidic environment of the stomach, gastrointestinal (GI) absorption and first-pass metabolism. Excipients and carriers that can be used to prepare parenteral formulations include without limitation solvents (e.g., aqueous solvents such as water, saline, physiological saline, buffered saline [e.g., PBS], balanced salt solutions [e.g., Ringer's BSS] and aqueous dextrose solutions), isotonic/iso-osmotic agents (e.g., salts [e.g., NaCl, KC1 and CaCl₂] and sugars [e.g., sucrose]), buffering agents and pH adjusters (e.g., sodium dihydrogen phosphate [monobasic sodium phosphate]/di sodium hydrogen phosphate [dibasic sodium phosphate], citric acid/sodium citrate and L-histidine/L-histidine HCl), and emulsifiers (e.g., non-ionic surfactants such as polysorbates [e.g., polysorbate 20 and 80] and poloxamers [e.g., poloxamer 188]). Peptide formulations and delivery systems are discussed in, e.g., A. J. Banga, Therapeutic Peptides and Proteins: Formulation, Processing, and Delivery Systems, 3rd Ed., CRC Press (Boca Raton, Florida) (2015). The excipients can optionally include one or more substances that increase peptide stability, increase peptide solubility, inhibit peptide aggregation or reduce solution viscosity, or any combination or all thereof. Such substances include without limitation hydrophilic amino acids (e.g., arginine and histidine), polyols (e.g., myo-inositol, mannitol and sorbitol), saccharides (e.g., glucose (including D-glucose [dextrose]), lactose, sucrose and trehalose}, osmolytes (e.g., trehalose, taurine, amino acids [e.g., glycine, sarcosine, alanine, proline, serine, b-alanine and g-aminobutyric acid], and betaines [e.g., trimethylglycine and trimethylamine N-oxide]), and non-ionic surfactants (e.g., alkyl polyglycosides, ProTek® alkylsaccarides (e.g., a monosaccharide [e.g., glucose] or a disaccharide [e.g., maltose or sucrose] coupled to a long-chain fatty acid or a corresponding long-chain alcohol), and polypropylene glycol/polyethylene glycol block co-polymers (e.g., poloxamers [e.g., Pluronic™F-68], and Genapol® PF-10 and variants thereof). Because such substances increase peptide solubility, they can be used to increase peptide concentration in a formulation. Higher peptide concentration in a formulation is particularly advantageous for subcutaneous administration, which has a limited volume of bolus administration (e.g., <about 1.5 mL). In addition, such substances can be used to stabilize peptides during the preparation, storage and reconstitution of lyophilized peptides. An exemplary parenteral formulation comprises a peptide product, mannitol, methionine, sodium thioglycolate, polysorbate 20, a pH adjuster (e.g., NaOH or/and HCl) and de-ionized water. Excipients of parenteral formulations that would be suitable for use with the modified peptides described herein (e.g., various combinations of excipients including NaCl and the like) are well-known and available to those of ordinary skill in the art.

For parenteral (e.g., subcutaneous, intravenous or intramuscular) administration, a sterile solution or suspension of a peptide product in an aqueous solvent containing one or more excipients can be prepared beforehand and can be provided in, e.g., a pre-filled syringe of a single-use pen or a pen with a dose counter. Alternatively, a peptide product can be dissolved or suspended in an aqueous solvent that can optionally contain one or more excipients prior to lyophilization (freeze-drying). Shortly prior to parenteral administration, the lyophilized peptide product stored in a suitable container (e.g., a vial) can be reconstituted with, e.g., sterile water that can optionally contain one or more excipients. In other embodiments, an agonist peptide product is administered intranasally. The nasal mucosa provides a big surface area, a porous endothelium, a highly vascular subepithelial layer and a high absorption rate, and hence allows for high bioavailability. An intranasal formulation can comprise a peptide product along with excipients, such as a solubility enhancer (e.g., propylene glycol), a humectant (e.g., mannitol or sorbitol), a buffer and water, and optionally a preservative (e.g., benzalkonium chloride), a mucoadhesive agent (e.g., hydroxyethylcellulose) or/and a penetration enhancer. An intranasal solution or suspension formulation can be administered to the nasal cavity by any suitable means, including but not limited to a dropper, a pipette, or spray using, e.g., a metering atomizing spray pump. Table 2 shows exemplary excipients of nasal-spray formulations.

TABLE 2
Exemplary excipients and carriers
of nasal and pulmonary formulations

| Dosage | |
|---|---|
| Form | Ingredients in Addition to a Peptide Product |
| nasal | microcrystalline cellulose, sodium carboxymethylcellulose, |
| spray | dextrose, water, and optionally a pH adjuster (e.g., HCl) |
| nasal | microcrystalline cellulose, carboxymethyl cellulose |
| spray | sodium, dextrose, polysorbate 80, disodium edetate, |
| | potassium sorbate, a pH adjuster (e.g., HCl), water, |
| | and optionally an alcohol (e.g., ethanol) |
| nasal | microcrystalline cellulose, carboxymethyl cellulose |
| spray | sodium, dextrose, polysorbate 80, benzalkonium chloride, |
| | phenylethyl alcohol, water, and optionally an alcohol |
| | (e.g., ethanol) |
| nasal | hypromellose, benzalkonium chloride, NaCl, EDTA, citric |
| spray | acid, sodium phosphate dibasic, water, and optionally |
| | an alcohol (e.g., ethanol) |
| inhalation | mannitol, glycine, sodium citrate and NaOH |
| (DPI) | |
| inhalation | lactose, starch, a starch derivative (e.g., |
| (DPI) | hydroxypropylmethyl cellulose) or polyvinylpyrrolidine, |
| | and optionally magnesium stearate or/and leucine |
| inhalation | a propellant (e.g., 1,1,1,2-tetrafluoroethane), a |
| (MPI) | surfactant (e.g., lecithin or oleic acid), and a |
| | co-solvent (e.g., ethanol) |
| inhalation | polysorbate 80, edetate disodium, sodium chloride, |
| (nebulizer) | pH buffering agents (e.g., citric acid/sodium |
| | citrate), and water |

In further embodiments, a peptide product is administered via a pulmonary route, such as by oral inhalation or nasal inhalation. Pulmonary administration of a drug can treat a lung disorder or/and a systemic disorder, as the lungs serve as a portal to the systemic circulation. Advantages of pulmonary drug delivery include, for example: 1) avoidance of first-pass metabolism; 2) fast drug action; 3) large surface area of the alveolar region for absorption, high permeability of the lungs (thin air-blood barrier), and profuse vasculature of the airways; and 4) reduced extracellular enzyme levels compared to the GI tract due to the large alveolar surface area. An advantage of oral inhalation over nasal inhalation includes deeper penetration/deposition of the drug into the lungs, although nasal inhalation can deliver the drug into systemic circulation transmucosally in the nasal cavity as well as in the lungs. Oral or nasal inhalation can be achieved by means of, e.g., a metered-dose inhaler (MDI), a nebulizer or a dry powder inhaler (DPI). For example, a peptide product can be formulated for aerosol administration to the respiratory tract by oral or nasal inhalation. The drug is delivered in a small particle size (e.g., between about 0.5 micron and about 5 microns), which can be obtained by micronization, to improve, e.g., drug deposition in the lungs and drug suspension stability. The drug can be provided in a pressurized pack with a suitable propellant, such as a hydrofluoroalkane (HFA, e.g., 1,1,1,2-tetrafluoroethane [HFA-134a]), a chlorofluorocarbon (CFC, e.g., dichlorodifluoromethane, trichlorofluoromethane or dichlorotetrafluoroethane), or a suitable gas (e.g., oxygen, compressed air or carbon dioxide). The drug in the aerosol formulation is dissolved, or more often suspended, in the propellant for delivery to the lungs. The aerosol can contain excipients such as a surfactant (which enhances penetration into the lungs by reducing the high surface tension forces at the air-water interface within the alveoli, may also emulsify, solubilize or/and stabilize the drug, and can be, e.g., a phospholipid such as lecithin) or/and a stabilizer, although the surfactant moiety of the peptide product can perform functions of a surfactant. For example, an MDI formulation can comprise a peptide product, a propellant (e.g., an HFA such as 1,1,1,2-tetrafluoroethane) and a co-solvent (e.g., an alcohol such as ethanol), and optionally a surfactant (e.g., a fatty acid such as oleic acid). The MDI formulation can optionally contain a dissolved gas (e.g., CO₂). After device actuation, the bursting of CO₂ bubbles within the emitted aerosol droplets breaks up the droplets into smaller droplets, thereby increasing the respirable fraction of drug. As another example, a nebulizer formulation can comprise a peptide product, a chelator or preservative (e.g., edetate disodium), an isotonicity agent (e.g., NaCl), pH buffering agents (e.g., citric acid/sodium citrate) and water, and optionally a surfactant (e.g., a Tween® such as polysorbate 80). The drug can be delivered by means of, e.g., a nebulizer or an MDI with or without a spacer, and the drug dose delivered can be controlled by a metering chamber (nebulizer) or a metering valve (MDI).

Table 2 shows exemplary MDI, nebulizer and DPI formulations. Metered-dose inhalers (also called pressurized metered-dose inhalers [pMDI]) are the most widely used inhalation devices. A metering valve delivers a precise amount of aerosol (e.g., about 20-100 pL) each time the device is actuated. MDIs typically generate aerosol faster than the user can inhale, which can result in deposition of much of the aerosol in the mouth and the throat. The problem of poor coordination between device actuation and inhalation can be addressed by using, e.g., a breath-actuated MDI or a coordination device. A breath-actuated MDI (e.g., Easi breathe®) is activated when the device senses the user's inspiration and discharges a drug dose in response. The inhalation flow rate is coordinated through the actuator and the user has time to actuate the device reliably during inhalation. In a coordination device, a spacer (or valved holding chamber), which is a tube attached to the mouthpiece end of the inhaler, serves as a reservoir or chamber holding the drug that is sprayed by the inhaler and reduces the speed at which the aerosol enters the mouth, thereby allowing for the evaporation of the propellant from larger droplets. The spacer simplifies use of the inhaler and increases the amount of drug deposited in the lungs instead of in the upper airways. The spacer can be made of an anti-static polymer to minimize electrostatic adherence of the emitted drug particles to the inner walls of the spacer. Nebulizers generate aerosol droplets of about 1-5 microns. They do not require user coordination between device actuation and inhalation, which can significantly affect the amount of drug deposited in the lungs. Compared to MDIs and DPIs, nebulizers can deliver larger doses of drug, albeit over a longer administration time. Examples of nebulizers include without limitation human-powered nebulizers, jet nebulizers (e.g., AeroEclipse® II BAN [breath-actuated], CompAIR™NE-C801 [virtual valve], PARI LC® Plus [breath-enhanced] and SideStream Plus [breath-enhanced]), ultrasonic wave nebulizers, and vibrating mesh nebulizers (e.g., Akita2® Apixneb, I-neb AAD System with metering chambers, MicroAir® NE-U22, Omron U22 and PARI eFlow® rapid). As an example, a pulsed ultrasonic nebulizer can aerosolize a fixed amount of the drug per pulse, and can comprise an opto-acoustical trigger that allows the user to synchronize each breath to each pulse. For oral or nasal inhalation using a dry powder inhaler (DPI), a peptide product can be provided in the form of a dry micronized powder, where the drug particles are of a certain small size (e.g., between about 0.5 micron and about 5 microns) to improve, e.g., aerodynamic properties of the dispersed powder and drug deposition in the lungs. Particles between about 0.5 micron and about 5 microns deposit by sedimentation in the terminal bronchioles and the alveolar regions. By contrast, the majority of larger particles (>5 microns) do not follow the stream of air into the many bifurcations of the airways, but rather deposit by impaction in the upper airways, including the oropharyngeal region of the throat. A DPI formulation can contain the drug particles alone or be blended with a powder of a suitable larger base/carrier, such as lactose, starch, a starch derivative (e.g., hydroxypropylmethyl cellulose) or polyvinylpyrrolidine. The carrier particles enhance flow, reduce aggregation, improve dose uniformity and aid in dispersion of the drug particles. A DPI formulation can optionally contain an excipient such as magnesium stearate or/and leucine that improves the performance of the formulation by interfering with inter-particle bonding (by anti-adherent action). The powder formulation can be provided in unit dose form, such as a capsule (e.g., a gelatin capsule) or a cartridge in a blister pack, which can be manually loaded or pre-loaded in an inhaler. The drug particles can be drawn into the lungs by placing the mouthpiece or nosepiece of the inhaler into the mouth or nose, taking a sharp, deep inhalation to create turbulent airflow, and holding the breath for a period of time (e.g., about 5-10 seconds) to allow the drug particles to settle down in the bronchioles and the alveolar regions. When the user actuates the DPI and inhales, airflow through the device creates shear and turbulence, inspired air is introduced into the powder bed, and the static powder blend is fluidized and enters the user's airways. There, the drug particles separate from the carrier particles due to turbulence and are carried deep into the lungs, while the larger carrier particles impact on the oropharyngeal surfaces and are cleared. Thus, the user's inspiratory airflow achieves powder de-agglomeration and aeroionisation, and determines drug deposition in the lungs. (While a passive DPI requires rapid inspiratory airflow to de agglomerate drug particles, rapid inspiration is not recommended with an MDI or nebulizer, since it creates turbulent airflow and fast velocity which increase drug deposition by impaction in the upper airways.) Compared to an MDI, a DPI (including a breath-activated DPI) may be able to deliver larger doses of drug, and larger-size drugs (e.g., macromolecules), to the lungs.

Lactose (e.g., alpha-lactose monohydrate) is the most commonly used carrier in DPI formulations. Examples of grades/types of lactose monohydrate for DPI formulations include without limitation DCL 11, Flowlac® 100, Inhalac® 230, Lactohale® 300, Lactopress® SD 250 (spray-dried lactose), Respitose® SV003 and Sorbolac® 400. A DPI formulation can contain a single lactose grade or a combination of different lactose grades. For example, a fine lactose grade like Lactohale® 300 or Sorbolac® 400 may not be a suitable DPI carrier and may need to be blended with a coarse lactose grade like DCL 11, Flowlac® 100, Inhalac® 230 or Respitose® SV003 (e.g., about a 1:9 ratio of fine lactose to coarse lactose) to improve flow.

Tables 3 and 4 show non-limiting examples of grades/types of lactose that can be used in DPI formulations. The distribution of the carrier particle sizes affects the fine particle fraction/dose (FPF or FPD) of the drug, with a high FPF being desired for drug delivery to the lungs. FPF/FPD is the respirable fraction/dose mass out of the DPI device with an aerodynamic particle size <5 microns in the inspiration air. High FPF, and hence good DPI performance, can be obtained from, e.g., DPI formulations having an approximately 1:9 ratio of fine lactose (e.g., Lactohale® 300) to coarse lactose (e.g., Respitose® SV003) and about 20% w/w overages to avoid deposition of the drug in the capsule shell or the DPI device and to deliver essentially all of the drug to the airways.

TABLE 3

| | | Range of Particle Sizes (μm) | | |
|---|---|---|---|---|
| Product | Type | 10% | 50% | 90% |
| Lactohale ® | LH200 | <9 | <69 | <141 |
| InhaLac ® | 230 | <35 | <93 | <138 |
| Respitose ® | ML001 | <4 | <43 | <146 |
| | ML003 | <4 | <35 | <106 |
| | SV003 | <30 | <59 | <90 |
| | SV004 | <32 | <61 | <93 |

TABLE 4

| | | Range of Particle Sizes | | | |
|---|---|---|---|---|---|
| Product | Type | <45 μm | <100 μm | <150 μm | <250 μm |
| Respitose ® | ML003 | 65% | 98% | 100% | NA |
| Rcspitosc ® | ML002 | 65% | 98% | NA | 100% |

Other carriers for DPI formulations include without limitation glucose, mannitol (e.g., crystallized mannitol [Pearlitol 110 C] and spray-dried mannitol [Pearlitol 100 SD]), maltitol (e.g., crystallized maltitol [Maltisorb P90]), sorbitol and xylitol. Most DPIs are breath-activated (“passive”), relying on the user's inhalation for aerosol generation. Examples of passive DPIs include without limitation Airmax®, Novolizer® and Otsuka DPI (compact cake). The air classifier technology (ACT) is an efficient passive powder dispersion mechanism employed in DPIs. In ACT, multiple supply channels generate a tangential airflow that results in a cyclone within the device during inhalation. There are also power-assisted (“active”) DPIs (based on, e.g., pneumatics, impact force or vibration) that use energy to aid, e.g., particle de-agglomeration. For example, the active mechanism of Exubera® inhalers utilizes mechanical energy stored in springs or compressed-air chambers. Examples of active DPIs include without limitation Actispire® (single-unit dose), Aspirair® (multi-dose), Exubera® (single-unit dose), MicroDose® (multi-unit dose and electronically activated), Omnihaler® (single-unit dose), Pfeiffer DPI (single-unit dose), and Spiros® (multi-unit dose). A peptide product can also be administered by other routes, such as orally. An oral formulation can contain a peptide product and conventional excipients known in the art, and optionally an absorption enhancer such as sodium V-[8-(2-hydroxybenzoyl) aminocaprylate] (SNAC). SNAC protects against enzymatic degradation via local buffering action and enhances GI absorption. An oral dosage form (e.g., a tablet, capsule or pill) can optionally have an enteric coating to protect its content from the strong acids and proteolytic enzymes of the stomach. In some embodiments, a peptide product is delivered from a sustained-release composition. As used herein, the term “sustained-release composition” encompasses sustained-release, prolonged-release, extended-release, delayed-release, slow-release and controlled-release compositions, systems and devices. In some embodiments, a sustained-release composition delivers a peptide product over a period of at least about 1 week, 2 weeks, 3 weeks, 1 month, 2 months, 3 months or longer. In some embodiments, a sustained-release composition is formulated as nanoparticles or microparticles composed of a biodegradable polymer and incorporating a peptide product. In certain embodiments, the biodegradable polymer comprises lactic acid or/and glycolic acid [e.g., an L-lactic acid-based copolymer, such as poly(L-lactide-co-glycolide) or poly(L-lactic acid-co- D,L-2-hydroxyoctanoic acid)]. In further embodiments, a sustained-release composition is in the form of a depot that is generated when a mixture of a peptide product and a polymer is injected into a subject intramuscularly or subcutaneously. In certain embodiments, the polymer is or comprises PEG, polylactic acid (PLA) or polyglycolic acid (PGA), or a copolymer thereof (e.g., PLGA or PLA-PEG).

A pharmaceutical composition can be presented in unit dosage form as a single dose wherein all active and inactive ingredients are combined in a suitable system, and components do not need to be mixed to form the composition to be administered. A unit dosage form generally contains a therapeutically effective dose of the drug but can contain an appropriate fraction thereof so that taking multiple unit dosage forms achieves the therapeutically effective dose. Examples of a unit dosage form include a tablet, capsule or pill for oral uptake; a solution in a pre-filled syringe of a single-use pen or a pen with a dose counter for parenteral (e.g., intravenous, subcutaneous or intramuscular) injection; and a capsule, cartridge or blister pre-loaded in or manually loaded into an inhaler. Alternatively, a pharmaceutical composition can be presented as a kit in which the active ingredient, excipients and carriers (e.g., solvents) are provided in two or more separate containers (e.g., ampules, vials, tubes, bottles or syringes) and need to be combined to form the composition to be administered. The kit can contain instructions for storing, preparing and administering the composition (e.g., a solution to be injected parenterally). A kit can contain all active and inactive ingredients in unit dosage form or the active ingredient and inactive ingredients in two or more separate containers and can contain instructions for administering or using the pharmaceutical composition to treat a medical condition disclosed herein. A kit can further contain a device for delivering the composition, such as an injection pen or an inhaler. In some embodiments, a kit contains a peptide product or a pharmaceutically acceptable salt thereof, or a pharmaceutical composition comprising the same, and instructions for administering or using the peptide product or the composition to treat a medical condition disclosed herein, such as insulin resistance, diabetes, obesity, metabolic syndrome or a cardiovascular disease, or a condition associated therewith (e.g., NASH or PCOS). In certain embodiments, the kit further contains a device for delivering the peptide product or the composition, such as an injection pen or an inhaler.

The disclosure further provides uses of the modified peptide products described herein to prevent and/or treat conditions associated with GLP1R, GIPR and/or GCGR, such as but not limited to insulin resistance, diabetes, obesity, metabolic syndrome and cardiovascular diseases, and conditions associated therewith, such as NASH and PCOS. In some embodiments, the modified peptide products can be used to treat hyperglycemia, insulin resistance, hyperinsulinemia, prediabetes, diabetes (including types 1 and 2, gestational and juvenile diabetes), diabetic complications, diabetic neuropathy, diabetic nephropathy, diabetic retinopathy, hyperlipidemia, hypercholesterolemia, hypertriglyceridemia, elevated blood levels of free fatty acids, obesity, metabolic syndrome, syndrome X, cardiovascular diseases (including coronary artery disease), atherosclerosis, acute cardiovascular syndrome, ischemia (including myocardial ischemia and cerebral ischemia/stroke), ischemia-reperfusion injury (including myocardial and cerebral IRI), infarction (including myocardial and cerebral infarction), angina, heart failure (e.g., congestive heart failure), peripheral vascular disease, thrombosis (e.g., deep vein thrombosis), embolism (e.g., pulmonary embolism), systemic inflammation (e.g., one characterized by elevated C-reactive protein blood level), and hypertension. The modified peptide products can achieve their therapeutic effects through various mechanisms, including stimulation of blood glucose-dependent insulin secretion, increase in insulin sensitivity, stimulation of fat burning and reduction of body weight. The modified peptide products can also promote, e.g., pancreatic beta-cell protection, cardioprotection and wound healing.

The peptide products described herein can be used to treat other conditions associated with insulin resistance or/and obesity (including chronic weight management). Other conditions associated with insulin resistance or/and obesity include without limitation arthritis (e.g., osteoarthritis), low back pain, breathing disorders (e.g., asthma, obesity hypoventilation syndrome [Pickwickian syndrome] and obstructive sleep apnea), dermatological disorders (e.g., diabetic ulcers, acanthosis nigricans, cellulitis, hirsutism, intertrigo and lymphedema), gastroenterological disorders (e.g., cholelithiasis [gallstone], gastroesophageal reflux disease [GERD] and gastroparesis), gout, hypercortisolism (e.g., Cushing's syndrome), kidney disorders (e.g., chronic kidney disease), liver disorders (e.g., fatty liver disease [FLD] including alcoholic and non-alcoholic FLD), neurological disorders (e.g., carpal tunnel syndrome, dementias [e.g., Alzheimer's disease and vascular dementia], meralgia paresthetica, migraines and multiple sclerosis), urological disorders (e.g., erectile dysfunction, hypogonadism and urinary incontinence), polycystic ovary syndrome, infertility, menstrual disorders, mood disorders (e.g., depression), and cancers (e.g., cancers of the endometrium, esophagus, colorectum, gallbladder, kidney, liver [e.g., hepatocellular carcinoma], pancreas and skin [e.g., melanoma], and leukemia). In certain embodiments, a modified peptide product described herein is used to treat polycystic ovary syndrome (PCOS). In other embodiments, a peptide product is used to treat chronic kidney disease (CKD), also known as chronic kidney/renal failure (CKF/CRF). The most common causes of CKD are diabetes and long-term, uncontrolled hypertension. In further embodiments, a modified peptide product described herein is used to treat fatty liver disease (FLD). In some embodiments, the FLD is non-alcoholic fatty liver disease (NAFLD). In certain embodiments, the NAFLD is non-alcoholic steatohepatitis (NASH). FLD, also known as hepatic steatosis, is characterized by excessive fat accumulation in the liver. FLD includes alcoholic fatty liver disease (AFLD) and NAFLD. Chronic alcoholism causes fatty liver due to production of toxic metabolites such as aldehydes during metabolism of alcohol in the liver. NAFLD is described below. FLD is associated with diabetes, obesity and metabolic syndrome. Fatty liver can develop into cirrhosis or a liver cancer (e.g., hepatocellular carcinoma [HCC]). Less than about 10% of people with cirrhotic AFLD develop HCC, but up to about 45% of people with NASH without cirrhosis may develop HCC. HCC is the most common type of primary liver cancer in adults and occurs in the setting of chronic liver inflammation. NAFLD is characterized by fatty liver that occurs when fat, in particular free fatty acids and triglycerides, accumulates in liver cells (hepatic steatosis) due to causes other than excessive alcohol consumption, such as nutrient overload, high caloric intake and metabolic dysfunction (e.g., dyslipidemia and impaired glucose control). A liver can remain fatty without disturbing liver function, but a fatty liver can progress to become NASH, a condition in which steatosis is accompanied by inflammation, hepatocyte ballooning and cell injury with or without fibrosis of the liver. Fibrosis is the strongest predictor of mortality from NASH. NAFLD can be characterized by steatosis alone; steatosis with lobular or portal inflammation but without ballooning; steatosis with ballooning but without inflammation; or steatosis with inflammation and ballooning. NASH is the most extreme form of NAFLD. NASH is a progressive disease, with about 20% of patients developing cirrhosis of the liver and about 10% dying from a liver disease, such as cirrhosis or a liver cancer (e.g., HCC). NAFLD is the most common liver disorder in developed countries, and NASH is projected to supplant hepatitis C as the major cause of liver transplant in the U.S. by 2020. About 12-25% of people in the U.S. have NAFLD, with NASH affecting about 2-5% of people in the U.S. NAFLD, including NASH, is associated with insulin resistance, obesity and metabolic syndrome. For instance, insulin resistance contributes to progression of fatty liver to hepatic inflammation and fibrosis and thus NASH. Furthermore, obesity drives and exacerbates NASH, and weight loss can alleviate NASH. Therefore, the peptide products described herein, including GLP-1 receptor (GLP1R) agonists, glucagon receptor (GCGR) agonists and dual GLP1R/GCGR agonists, can be used to treat NAFLD, including NASH. In method of treatment for chronic weight management of a human being with a body mass index (BMI kg/m²) of at least 25 (including chronically overweight (BMI of 25 or greater) “chronic obesity” meaning obesity (BMI of 30 or greater)) lasting more than one year or resulting in an obesity-related condition such as but not limited to insulin resistance, diabetes, metabolic syndrome, and/or cardiovascular disease) by inducing weight loss in the human being, the method comprising administering to the human being a once weekly therapeutic effective amount of a pharmaceutical dosage formulation comprising any of the modified peptides disclosed herein, wherein the weight of the human being is reduced by at least 5-10% from baseline at week 12. In certain other embodiments, a modified peptide of this disclosure may be used as an adjunct treatment for chronic weight management in obese (i.e., BMI of 30 or greater) or overweight (i.e., BMI of 25 of greater) subjects in combination with a reduced calorie diet and/or increased physical activity. In some embodiments, the modified peptide products used to treat a condition associated with insulin resistance or/and obesity disclosed herein, such as NAFLD (e.g., NASH) or PCOS, are selected from the modified peptide products of SEQ. ID. NOS: 1-29, and/or derivatives thereof, and pharmaceutically acceptable salts thereof.

In some embodiments, the present modified peptide(s) can be used to control blood glucose with reduction of one or more adverse events (i.e., an unexpected event that negatively impacts patient and/or animal welfare) as compared to an agonist with unbalanced affinity for GLP-1R and GCGR (e.g., semaglutide). Exemplary, non-limiting adverse events can include nausea, vomiting, diarrhea, abdominal pain and/or constipation. Adverse events may also include any known to those of ordinary skill in the art, such as those listed in industry resources and/or otherwise known to those of ordinary skill in the art (see, e.g., Medical Dictionary for Regulatory Activities (MedDRA) (Pharm., Med. Transl. Med. 2018) and/or Clark, M. J. Biomed. Inf., 54, April 2015, pp. 167-173). Such adverse events can be determined in humans using standard techniques as are typically used in clinical trials (e.g., doctor visit, surveys/questionnaires). As compared to the frequency and/or severity of such an adverse event that occurs upon administration of an agonist with unbalanced affinity for GLP-1R and GCGR (e.g., semaglutide) to a subject, the modified peptides of this disclosure (e.g., any of SEQ ID NOS. 1-29, and/or derivatives thereof) can decrease such frequency and/or severity thereof by, e.g., 20%, 40%, 50%, 60%, 70%, 80%, 90% of higher (up to 100%). In some embodiments, the modified peptides of this disclosure (e.g., any of SEQ ID NOS. 1-29, and/or derivatives thereof) do not cause any adverse events.

A present modified peptide product can be administered by any suitable route for treatment of a condition disclosed herein. Potential routes of administration of a peptide product include without limitation oral, parenteral (including intradermal, subcutaneous, intramuscular, intravascular, intravenous, intra-arterial, intraperitoneal, intracavitary and topical), and topical (including transdermal, transmucosal, intranasal (e.g., by nasal spray or drop), ocular (e.g., by eye drop), pulmonary (e.g., by oral or nasal inhalation), buccal, sublingual, rectal (e.g., by suppository), and vaginal (e.g., by suppository)). In some embodiments, a peptide product is administered parenterally, such as subcutaneously, intravenously or intramuscularly. In other embodiments, a peptide product is administered by oral inhalation or nasal inhalation or insufflation. The therapeutically effective amount and the frequency of administration of, and the length of treatment with, a peptide product to treat a condition disclosed herein may depend on various factors, including the nature and severity of the condition, the potency of the compound, the route of administration, the age, body weight, general health, gender and diet of the subject, and the response of the subject to the treatment, and can be determined by the treating physician. In some embodiments, a peptide product is administered parenterally (e.g., subcutaneously (sc), intravenously (iv) or intramuscularly (im)) in a dose from about 0.01 mg to about 0.1, 1, 5 or 10 mg, or about 0.1-1 mg or 1-27 mg, over a period of about one week for treatment of a condition disclosed herein (e.g., one associated with insulin resistance or/and obesity, such as NASH or PCOS). In further embodiments, a peptide product is administered parenterally (e.g., sc, iv or im) in a dose of about 0.1-0.5 mg, 0.5-1 mg, 1-5 mg or 5-10 mg over a period of about one week. In certain embodiments, a peptide product is administered parenterally (e.g., subcutaneously (SC), intravenous (IV) or intramuscular (IM)) in a dose of about 0.1-1 mg, or about 0.1-0.5 mg or 0.5-1 mg, over a period of about one week. One of skill in the art understands that an effective dose in a mouse, or other pre-clinical animal model, may be scaled for a human. In that way, through allometric scaling (also referred to as biological scaling) a dose in a larger animal may be extrapolated from a dose in a mouse to obtain an equivalent dose based on body weight or body surface area of the animal.

A peptide product can be administered in any suitable frequency for treatment of a condition disclosed herein (e.g., one associated with insulin resistance or/and obesity, such as NASH or PCOS). In some embodiments, a modified peptide product is administered, e.g., sc or iv once a day, once every two days, once every three days, twice a week, once a week or once every two weeks. In certain embodiments, a peptide product is administered, e.g., SC, IV, or IM once a week. A modified peptide product can be administered at any time of day convenient to the patient. A modified peptide product can be taken substantially with food (e.g., with a meal or within about 1 hour or 30 minutes before or after a meal) or substantially without food (e.g., at least about 1 or 2 hours before or after a meal). The length of treatment of a medical condition with a modified peptide product can be based on, e.g., the nature and severity of the condition and the response of the subject to the treatment and can be determined by the treating physician. In some embodiments, a modified peptide product is administered chronically to treat a condition disclosed herein, such as at least about 2 months, 3 months, 6 months, 1 year, 1.5 years, 2 years, 3 years, 5 years, 10 years or longer. A modified peptide product can also be taken pro re nata (as needed) until clinical manifestations of the condition disappear or clinical targets are achieved, such as blood glucose level, blood pressure, blood levels of lipids, body weight or body mass index, waist-to-hip ratio or percent body fat, or any combination thereof If clinical manifestations of the condition re-appear or the clinical targets are not maintained, administration of the modified peptide product can resume. The disclosure provides a method of treating a medical condition described herein, comprising administering to a subject in need of treatment a therapeutically effective amount of a peptide product described herein or a pharmaceutically acceptable salt thereof, or a pharmaceutical composition comprising the same. The disclosure further provides a peptide product described herein or a pharmaceutically acceptable salt thereof, or a composition comprising the same, for use as a medicament. In addition, the disclosure provides for the use of a peptide product described herein or a pharmaceutically acceptable salt thereof in the preparation of a medicament. The medicament containing the peptide product can be used to treat any medical condition described herein. The peptide product can optionally be used in combination with one or more additional therapeutic agents.

A modified peptide product described herein can be administered as the sole active agent, or optionally be used in combination with one or more other modified peptide products, and/or additional therapeutic agents to treat any disorder disclosed herein, such as insulin resistance, diabetes, obesity, metabolic syndrome or a cardiovascular disease, or any condition associated therewith, such as NASH or PCOS. In some embodiments, the one or more additional therapeutic agents are selected from antidiabetic agents, anti-obesity agents (including lipid-lowering agents and pro-satiety agents), anti-atherosclerotic agents, anti-inflammatory agents, antioxidants, antifibrotic agents, anti-hypertensive agents, and combinations thereof. Antidiabetic agents include without limitation: AMP-activated protein kinase (AMPK) agonists, including biguanides (eg., buformin and metformin); peroxisome proliferator-activated receptor gamma (PPAR-γ) agonists, including thiazolidinediones (e.g., balaglitazone, ciglitazone, darglitazone, englitazone, lobeglitazone, netoglitazone, pioglitazone, rivoglitazone, rosiglitazone and troglitazone), MSDC-0602K and saroglitazar (dual PPAR-α/γ agonist); glucagon-like peptide-1 (GLP-1) receptor agonists, including exendin-4, albiglutide, dulaglutide, exenatide, liraglutide, lixisenatide, semaglutide, taspoglutide, CNT0736, CNT03649, HM11260C (LAPS-Exendin), NN9926 (OG9S7GT), TT401 and ZYOG1; dipeptidyl peptidase 4 (DPP-4) inhibitors, including alogliptin, anagliptin, dutogliptin, evogliptin, gemigliptin, gosogliptin, linagliptin, omarigliptin, saxagliptin, septagliptin, sitagliptin, teneligliptin, trelagliptin and vildagliptin; sodium-glucose transport protein 2 (SGLT2) inhibitors, including canagliflozin (also inhibits SGLT1), dapagliflozin, empagliflozin, ertugliflozin, ipragliflozin, luseogliflozin, remogliflozin etabonate, sotagliflozin (also inhibits SGLT1) and tofogliflozin; blockers of ATP-dependent K⁺ (KA_{TP}) channels on pancreatic beta cells, including rneglitinides (e.g., mitiglinide, nateglinide and repagiinide) and sulfonylureas (including first generation (e.g., acetohexamide, carbutamide, chlorpropamide, giycyclamide [tolhexamide], metahexamide, tolazamide and tolbutamide) and second generation (e.g., glibenclamide, glyburide, glibornuride, gliclazide, glimepiride, glipizide, gliquidone, glisoxepide and glyclopyramide); insulin and analogs thereof, including fast-acting insulin (e.g., insulin aspari insulin glulisine and insulin lispro), intermediate-acting insulin (e.g., NPH insulin), and long-acting insulin (e.g., insulin degludec, insulin detemir and insulin glargine); and/or, analogs, derivatives and salts thereof In certain embodiments, the antidiabetic agent is or includes a biguanide (e.g., metformin), a thiazolidinedione (e.g., pioglitazone or rosiglitazone) or a SGLT2 inhibitor (e.g., empagliflozin or tofogliflozin), or any combination thereof. Anti-obesity agents include, but are not limited to: appetite suppressants (anorectics), including amphetamine, dexamphetamine, amfepramone, clobenzorex, mazindol, phentermine (with or without topiramate) and lorcaserin; pro-satiety agents, including ciliary neurotrophic factor (e.g., axokine) and longer-acting analogs of amylin, calcitonin, cholecystokinin (CCK), GLP-1, leptin, oxyntomodulin, pancreatic polypeptide (PP), peptide YY (PYY) and neuropeptide Y (NPY); lipase inhibitors, including caulerpenyne, cetilistat, ebelactone A and B, esterastin, lipstatin, orlistat, percyquinin, panclicin A-E, valilactone and vibralactone; antihyperlipidemic agents; and analogs, derivatives and salts thereof. Antihyperlipidemic agents include without limitation: HMG-CoA reductase inhibitors, including statins {e.g., atorvastatin, cerivastatin, fluvastatin, mevastatin, monacolins (e.g., monacolin K (lovastatin), pitavastatin, pravastatin, rosuvastatin and simvastatin} and flavanones (e.g., naringenin); squalene synthase inhibitors, including lapaquistat, zaragozic acid and RPR-107393; acetyl-CoA carboxylase (ACC) inhibitors, including anthocyanins, avenaciolides, chloroacetylated biotin, cyclodim, diclofop, haloxyfop, soraphens (e.g., soraphen A₁ₐ), 5-(tetradecyloxy)-2-furancarboxylic acid (TOFA), CP-640186, GS-0976, NDI-010976; 7-(4-propyloxy-phenylethynyl)-3,3 -dimethyl-3,4dihydro-2H-benzo [b][1,4]dioxepine; N-ethyl-N′-(3-{[4-(3,3-dimethyl-1-oxo-2-oxa-7-azaspiro[4.5]dec-7-yl) piperidin-1-yl]-carbonyl}-1-benzothien-2-yl)urea; 5-(3-acetamidobut-1-ynyl)-2-(4-propyloxyphenoxy) thiazole; and 1-(3-{[4-(3,3-dimethyl-1-oxo-2-oxa-7-azaspiro[4.5]dec-7-yl) piperidin-1-yl]-carbonyl}-5-(pyridin-2-yl)-2-thienyl)-3-ethylurea; PPAR-α agonists, including fibrates (e.g., bezafibrate, ciprofibrate, clinofibrate, clofibric acid, clofibrate, aluminum clofibrate [alfibrate], clofibride, etofibrate, fenofibric acid, fenofibrate, gemfibrozil, ronifibrate and simfibrate), isoflavones (e.g., daidzein and genistein), and perfluoroalkanoic acids (e.g., perfluorooctanoic acid and perfluorononanoic acid); PPAR-δ agonists, including elafibranor (dual PPAR-α/γ agonist), GFT505 (dual PPAR-α/γ agonist), GW0742, GW501516 (dual PPAR-β/δ agonist), sodelglitazar (GW677954), MBX-8025, and isoflavones (e.g., daidzein and genistein); PPAR-γ agonists, including thiazolidinediones {supra), saroglitazar (dual PPAR-α/γ agonist), 4-oxo-2-thioxothiazolines (e.g., rhodanine), berberine, honokiol, perfluorononanoic acid, cyclopentenone prostaglandins (e.g., cyclopentenone 15-deoxy-A-prostaglandin J₂[15d-PGJ₂]), and isoflavones (e.g., daidzein and genistein); liver X receptor (LXR) agonists, including endogenous ligands (e.g., oxysterols such as 22(i?)-hydroxycholesterol, 24(A)-hydroxy cholesterol, 27-hydroxycholesterol and cholestenoic acid) and synthetic agonists (e.g., acetyl-podocarpic dimer, hypocholamide, A(X-dimethyl-3b-hydroxy-cholenamide [DMHCA], GW3965 and T0901317); retinoid X receptor (RXR) agonists, including endogenous ligands (e.g., 9-cis-retinoic acid) and synthetic agonists (e.g., bexarotene, AGN 191659, AGN 191701, AGN 192849, BMS649, LG100268, LG100754 and LGD346); inhibitors of acyl-CoA cholesterol acyltransferase (ACAT, aka sterol G-acyl transferase [SOAT], including ACAT1 [SOAT1] and ACAT2 [SOAT2]), including avasimibe, pactimibe, pellitorine, terpendole C and flavanones (e.g., naringenin); inhibitors of stearoyl-CoA desaturase-1 (SCD-1, aka stearoyl-CoA delta-9 desaturase) activity or expression, including aramchol, CAY-10566, CVT-11127, SAR-224, SAR-707, XEN-103; 3-(2-hydroxyethoxy)-4-methoxy-N-[5-(3-trifluoromethylbenzyl) thiazol-2-yl]benzamide and 4-ethylamino-3-(2-hydroxyethoxy)-N-[5-(3-trifluoromethylbenzyl) thiazol-2-yl]benzamide; 1′-{6-[5-(pyridin-3-ylmethyl)-1,3,4-oxadiazol-2-yl]pyridazin-3-yl}-5-(trifluoromethyl)-3,4-dihydrospiro[chromene-2,4′-piperidine]; 5-fluoro-1′-{6-[5-(pyridin-3-ylmethyl)-1,3,4-oxadiazol-2-yl]pyridazin-3-yl}-3,4-dihydrospiro[chromene-2,4′-piperidine]; 6-[5-(cyclopropylmethyl)-4,5-dihydro-1′H,3H-spiro [1,5-benzoxazepine-2,4′-piperidin]-1′-yl]-N-(2-hydroxy-2-pyridin-3-ylethyl)pyridazine-3-carboxamide; 6-[4-(2-methylbenzoyl)piperidin-1-yl]pyridazine-3-carboxylic acid (2-hydroxy-2-pyridin-3-ylethyl) amide; 4-(2-chlorophenoxy)-N-[3-(methylcarbamoyl)phenyl]piperidine-1-carboxamide; the cis-9,trans-11 isomer and the trans-10,cis-12 isomer of conjugated linoleic acid, substituted heteroaromatic compounds disclosed in WO 2009/129625 A1, anti-sense polynucleotides and peptide-nucleic acids (PNAs) that target mRNA for SCD-1, and SCD-1-targeting siRNAs; cholesterylester transfer protein (CETP) inhibitors, including anacetrapib, dalcetrapib, evacetrapib, torcetrapib and AMG 899 (TA-8995); inhibitors of microsomal triglyceride transfer protein (MTTP) activity or expression, including implitapide, lomitapide, dirlotapide, mitratapide, CP-346086, JTT-130, SLx-4090, anti-sense polynucleotides and PNAs that target mRNA for MTTP, MTTP -targeting microRNAs (e.g., miRNA-30c), and MTTP-targeting siRNAs; GLP-1 receptor agonists; fibroblast growth factor 21 (FGF21) and analogs and derivatives thereof, including BMS-986036 (pegylated FGF21); inhibitors of pro-protein eonvertase subtilisin/kexin type 9 (PCSK9) activity or expression, including berberine (reduces PC8K9 level), annexin A2 (inhibits PCSK9 activity), anti-PCSK9 antibodies (e.g., alirocumab, bococizumab, evolocumab, LGT-209, LY3015014 and RG7652), peptides that mimic the epidermal growth factor-A (EGF-A) domain of the LDL receptor which binds to PCSK9, PCSK9-binding adnectins (e.g., BMS-962476), anti-sense polynucleotides and PNAs that target mRNA for PCSK9, and PCSK9-targeting siRNAs (e.g, inclisiran [ALN-PCS] and ALN-PCS02); apolipoprotein mimetic peptides, including apoA-I mimetics (e.g., 2F, 3F, 3F-1, 3F-2, 3F-14, 4F, 4F-P-4F, 4F-IHS-4F, 4F2, 5F, 6F, 7F, 18F, 5A, 5A-C1, 5A-CH1, 5A-CH2, 5A-H1, 18 A, 37pA [18A-P-18A], ELK, ELK-1A, ELK-1F, ELK-1K1A1E, ELK-1L1K, ELK-1W, ELK-2A, ELK-2A2K2E, ELK-2E2K, ELK-2F, ELK-3 E3EK, ELK-3E3K3A, ELK-3E3LK, ELK-PA, ELK-P2A, ELKA, ELKA-CH2, ATI-5261, CS-6253, ETC-642, FAMP, FREL and KRES and apoE mimetics (e.g., Ac-hEl8A-NH₂, AEM-28, Ac-[R]hEl 8 A-NH₂, AEM-28-14, EpK, hEp, mR18L, COG-112, COG-133 and COG-1410); omega-3 fatty acids, including docosahexaenoic acid (DHA), docosapentaenoic acid (DPA), eicosapentaenoic acid (EPA), a-linolenic acid (ALA), fish oils (which contain, e.g., DHA and EPA), and esters (e.g., glyceryl and ethyl esters) thereof; and analogs, derivatives and salts thereof. In certain embodiments, the anti-obesity agent is or includes a lipase inhibitor (e.g., orlistat) or/and an antihyperlipidemic agent (e.g., a statin such as atorvastatin, or/and a fibrate such as fenofibrate). Antihypertensive agents include without limitation: antagonists of the renin-angiotensin-aldosterone system (RAAS), including renin inhibitors (e.g., aliskiren), angiotensin-converting enzyme (ACE) inhibitors (e.g., benazepril, captopril, enalapril, fosinopril, lisinopril, moexipril, perindopril, quinapril, ramipril and trandolapril), angiotensin II receptor type 1 (ATII1) antagonists (e.g., azilsartan, candesartan, eprosartan, fimasartan, irbesartan, losartan, olmesartan medoxomil, olmesartan, telmisartan and valsartan), and aldosterone receptor antagonists (e.g., eplerenone and spironolactone); diuretics, including loop diuretics (e.g., bumetanide, ethacrynic acid, furosemide and torsemide), thiazide diuretics (e.g., bendroflumethiazide, chlorothiazide, hydrochlorothiazide, epitizide, methyclothi azide and polythiazide), thiazide-like diuretics (e.g., chlorthalidone, indapamide and metolazone), cicletanine (an early distal tubular diuretic), potassium-sparing diuretics (e.g., amiloride, eplerenone, spironolactone and triamterene), and theobromine; calcium channel blockers, including dihydropyridines (e.g., amlodipine, levamlodipine, cilnidipine, clevidipine, felodipine, isradipine, lercanidipine, nicardipine, nifedipine, nimodipine, nisoldipine and nitrendipine) and non-dihydropyri dines (e.g., diltiazem and verapamil); α₂-adrenoreceptor agonists, including clonidine, guanabenz, guanfacine, methyldopa and moxonidine; α1-adrenoreceptor antagonists (alpha blockers), including doxazosin, indoramin, nicergoline, phenoxybenzamine, phentolamine, prazosin, terazosin and tolazoline; β-adrenoreceptor (β1 or/and β₂) antagonists (beta blockers), including atenolol, betaxolol, bisoprolol, carteolol, carvedilol, labetalol, metoprolol, nadolol, nebivolol, oxprenolol, penbutolol, pindolol, propranolol and timolol; mixed alpha/beta blockers, including bucindolol, carvedilol and labetalol; endothelin receptor antagonists, including selective ETA receptor antagonists (e.g., ambrisentan, atrasentan, edonentan, sitaxentan, zibotentan and BQ-123) and dual ETA/ETB antagonists (e.g., bosentan, macitentan and tezosentan); other vasodilators, including hydralazine, minoxidil, theobromine, sodium nitroprusside, organic nitrates (e.g., isosorbide mononitrate, isosorbide dinitrate and nitroglycerin, which are converted to nitric oxide in the body), endothelial nitric oxide synthase (eNOS) stimulators (e.g., cicletanine), activators of soluble guanylate cyclase (e.g., cinaciguat and riociguat), phosphodiesterase type 5 (PDE5) inhibitors (e.g., avanafil, benzamidenafil, dasantafil, dynafil, lodenafil, mirodenafil, sildenafil, tadalafil, udenafil, vardenafil, dipyridamole, papaverine, propentofylline, zaprinast and T-1032), prostaglandin Ei (alprostadil) and analogs thereof (e.g., limaprost amd misoprostol), prostacyclin and analogs thereof (e.g., ataprost, beraprost [e.g., esuberaprost], 5,6,7-trinor-4,8-inter-w-phenylene-9-fluoro-PG1₂, carbacyclin, isocarbacyclin, clinprost, ciprostene, eptaloprost, cicaprost, iloprost, pimilprost, SM-10906 (des-methyl pimilprost), naxaprostene, taprostene, treprostinil, CS-570, OP-2507 and TY-11223), non prostanoid prostacyclin receptor agonists (e.g., 1-phthalazinol, ralinepag, selexipag, ACT-333679 [MRE-269, active metabolite of selexipag], and TRA-418), phospholipase C (PLC) inhibitors, and protein kinase C (PKC) inhibitors (e.g., BIM-1, BIM-2, BIM-3, BIM-8, chelerythrine, cicletanine, gossypol, miyabenol C, myricitrin, ruboxistaurin and verbascoside; minerals, including magnesium and magnesium sulfate; and analogs, derivatives and salts thereof. In certain embodiments, the antihypertensive agent is or includes a thiazide or thiazide like diuretic (e.g., hydrochlorothiazide or chlorthalidone), a calcium channel blocker (e.g., amlodipine or nifedipine), an ACE inhibitor (e.g., benazepril, captopril or perindopril) or an angiotensin II receptor antagonist (e.g., olmesartan medoxomil, olmesartan, telmisartan or valsartan), or any combination thereof In some embodiments, a peptide product described herein is used in combination with one or more additional therapeutic agents to treat NAFLD, such as NASH. In some embodiments, the one or more additional therapeutic agents are selected from antidiabetic agents, anti-obesity agents, anti-inflammatory agents, antifibrotic agents, antioxidants, anti hypertensive agents, and combinations thereof. Therapeutic agents that can be used to treat NAFLD (e.g., NASH) include without limitation: PPAR agonists, including PPAR-δ agonists (e.g., MBX-8025, elafibranor [dual PPAR-α/δ agonist] and GW501516[ dual PPAR-β/δ agonist]) and PPAR-γ agonists (e.g., thiazolidinediones such as pioglitazone, and saroglitazar [dual PPAR-α/γ agonist])—PPAR-δ and -γ agonism increases insulin sensitivity, PPAR-α agonism reduces liver steatosis and PPAR-δ agonism inhibits activation of macrophages and Kupffer cells; farnesoid X receptor (FXR) agonists, such as obeticholic acid and nonsteroidal FXR agonists like GS-9674 reduce liver gluconeogenesis, lipogenesis, steatosis and fibrosis; fibroblast growth factor 19 (FGF19) and analogs and derivatives thereof, such as NGM-282-FGF19 analogs reduce liver gluconeogenesis and steatosis; fibroblast growth factor 21 (FGF21) and analogs and derivatives thereof, such as BMS-986036 (pegylated FGF21)-FGF21 analogs reduce liver steatosis, cell injury and fibrosis; HMG-CoA reductase inhibitors, including statins (e.g., rosuvastatin)-statins reduce steatohepatitis and fibrosis; ACC inhibitors, such as NDI-010976 (liver-targeted) and GS-0976-ACC inhibitors reduce de novo lipogenesis and liver steatosis; SCD-1 inhibitors, such as aramchol-SCD-1 inhibitors reduce liver steatosis and increase insulin sensitivity; SGLT2 inhibitors, such as canagliflozin, ipragliflozin and luseogliflozin-SGLT2 inhibitors reduce body weight, liver ALT level and fibrosis; antagonists of CCR2 or/and CCR5, such as cenicriviroc-antagonists of CCR2 (binds to CCL2[MCP1]) and CCR5 (binds to CCL5 [RANTES]) inhibit activation and migration of inflammatory cells (e.g., macrophages) to the liver and reduce liver fibrosis; apoptosis inhibitors, including apoptosis signal-regulating kinase 1 (ASK1) inhibitors (e.g., selonsertib) and caspase inhibitors (e.g., emricasan [pan-caspase inhibitor])-apoptosis inhibitors reduce liver steatosis and fibrosis; lysyl oxidase-like 2 (LOXL2) inhibitors, such as simtuzumab-LOXL2 is a key matrix enzyme in collagen formation and is highly expressed in the liver; galectin-3 inhibitors, such as GR-MD-02 and TD139-galectin-3 is critical for development of liver fibrosis; antioxidants, including vitamin E (e.g., a-tocopherol) and scavengers of reactive oxygen species (ROS) and free radicals (e.g., cysteamine, glutathione, melatonin and pentoxifylline [also anti-inflammatory via inhibition of TNF-a and phosphodiesterases])-vitamin E reduces liver steatosis, hepatocyte ballooning and lobular inflammation; and, analogs, derivatives and salts thereof In some embodiments, a peptide product described herein is used in conjunction with a PPAR agonist (e.g., a PPAR-δ agonist such as elafibranor or/and a PPAR-γ agonist such as pioglitazone), a HMG-CoA reductase inhibitor (e.g., a statin such as rosuvastatin), an FXR agonist (e.g., obeticholic acid) or an antioxidant (e.g., vitamin E), or any combination thereof, to treat NAFLD (e.g., NASH). In certain embodiments, the one or more additional therapeutic agents for treatment of NAFLD (e.g., NASH) are or include vitamin E or/and pioglitazone. Other combinations may also be used as would be understood by those of ordinary skill in the art.

Pharmacokinetic (“PK”) parameters can be estimated using Phoenix® WinNonlin® version 8.1 or higher (Certara USA, Inc., Princeton, New Jersey). A non-compartmental approach consistent with the extravascular route of administration can be used for parameter estimation. The individual plasma concentration-time data can be used for pharmacokinetic calculations. In addition to parameter estimates for individual animals, descriptive statistics (e.g. mean, standard deviation, coefficient of variation, median, min, max) can be determined, as appropriate. Concentration values that are below the limit of quantitation can be treated as zero for determination of descriptive statistics and pharmacokinetic analysis. Embedded concentration values that are below the limit of quantitation can be excluded from pharmacokinetic analysis. All parameters can be generated from individual modified peptide (or derivatives and/or metabolites thereof) concentrations in plasma from test article-treated groups on the day of dosing (Day 1). Parameters can be estimated using nominal dose levels, unless out of specification dose formulation analysis results are obtained, in which case actual dose levels can be used. Parameters can be estimated using nominal sampling times; if bioanalytical sample collection deviations are documented, actual sampling times can be used at the affected time points. Bioanalytical data can be used as received for the pharmacokinetic analysis and can be presented in tables and figures in the units provided. Pharmacokinetic parameters can be calculated and presented in the units provided by the analytical laboratory (the order of magnitude can be adjusted appropriately for presentation in the report, e.g., h*ng/mL converted to h *μg/mL). Descriptive statistics (e.g., mean, standard deviation, coefficient of variation, median, min, max) and pharmacokinetic parameters can be determined to three significant figures, as appropriate. Additional data handling items can be documented as needed. PK parameters to be determined, as data permit, can include but are not limited to the following: Cₘₐₓ: Maximum observed concentration; DN Cₘₐₓ: dose normalized maximum concentration, calculated as Cₘₐₓ/dose; Tₘₐₓ: time of maximum observed concentration; AUC₀₋₄: area under the curve from time 0 to the time of the last measurable concentration, calculated using the linear trapezoidal rule; AUC₀₋₉₆: area under the curve from time 0 to hour 96, calculated using the linear trapezoidal rule; DN AUC₀₋₉₆: dose normalized AUC₀₋₉₆, calculated as AUC₀₋₉₆/dose; AUC_{0-inf}: area under the curve from time 0 to infinity (Day 1 only), calculated as AUC_{0-inf}=AUC₀₋₄+Cₜ/λ_{z}, where Cₜ is the last observed quantifiable concentration and λ_{z} is the elimination rate constant; t_{1/2}: elimination half-life, calculated as ln(2)/λ_{z}. Additional parameters and comparisons (e.g., sex ratios, dose proportionality ratios, etc.) can also be determined, as would be understood by those of ordinary skill in the art.

In some embodiments, this disclosure provides pharmaceutical dosage formulation(s) comprising at least one modified peptide with affinity for glucagon-like peptide 1 receptor (GLP-1R), GIPR and/or glucagon receptor (GCGR) wherein: the peptide is modified with a hydrophobic surfactant; the dosage is configured to control blood glucose and/or induce weight loss, with reduction of one or more adverse events as compared to the parent or unmodified peptide, the adverse events being selected from nausea, vomiting, diarrhea, abdominal pain and constipation, upon administration to a mammal. In some embodiments, the modified peptide is any one of SEQ ID NOS: 1-29, and/or a derivative thereof, or a combination thereof. In some embodiments, the modified peptide has about equal affinity for GLP-1R and GCGR. In some embodiments, administration of the modified peptide to a mammal, as compared to administration of an approximate equimolar dosage of semaglutide, results in: lower blood glucose at about 48 or 96 hours following administration (optionally at least about any of 10, 20, 30, 40, or 50% lower, preferably at least about 50% lower); lower blood glucose at about 72 hours following administration (optionally at least about any of 10, 20, 30, 40, 50, 60, 70, 80, 90, or 100% lower, preferably at least about 100% lower); and/or, lower blood glucose at about 120 hours following administration. In some embodiments, administration of the modified peptide to a mammal, as compared to administration of an approximate equimolar dosage of semaglutide, induces whole-body weight loss; and/or, induces liver weight loss. In some embodiments, administration of the modified peptide to a mammal, as compared to administration of an approximate equimolar dosage of semaglutide, exhibits a lower Cmax (optionally at least about any of 10, 20, 30, 40, 50% lower, preferably at least about 50% lower); exhibits approximately equal or greater Tmax (optionally at least about any of 10, 20, 30, 40, 50, 60, 70, 80, 90, or 100% greater, preferably at least about 100% greater); exhibits a similar AUC_{(0-inf)} (optionally at least about any of 50, 60, 70, 80, 90, 95, 100% thereof, preferably at least about 80-90% thereof, such as about 85-93% thereof); exhibits about an equal or higher T_{1/2(hr)} (optionally at least about any of 10, 20, 30, 40, 50, 60, 70, 80, 90, or 100% thereof, preferably at least about 50 or 75% thereof, such as about 50-75% thereof); exhibits a prolonged MRT (hr) (optionally at least about any of 10, 20, 30, 40, or 50% higher, preferably at least about 25% higher); exhibits a protracted PK/PD profile; exhibits equal or greater glucoregulatory effects; induces greater whole-body weight loss, optionally about twice thereof; induces lower body fat mass, optionally about any of 10, 20, 30, 40, 50, 60, 70, 80, 90, or 100% lower, preferably at least about 100% lower); and/or, when administered to treat NASH induces increased whole-body weight reduction, liver weight loss, improved NAS score, improved hepatosteatosis, improved ballooning, improved col1A1 staining, improved ALT, improved liver TG/TC, and improved plasma TG/TC. In some embodiments, administration of the modified peptide to a mammal, as compared to administration of an approximate equimolar dosage of semaglutide, results in greater loss in body weight by approximately 14 days following administration of the dosage formulation (optionally at least about 10, 20, 30, 40 or 50% greater, preferably at least about 15% greater); and/or, greater loss in body weight by approximately 20-28 days following administration of the dosage formulation (optionally at least about any of 10, 20, 30, 40, or 50% greater, preferably at least about 25% greater). In some embodiments, administration of the modified peptide to a mammal, as compared to administration of an approximate equimolar dosage of semaglutide, results in weight loss in an obese mammal sufficient to return the mammal the normal weight range of a lean normal mammal.

“Reducing,” or “reduction of” adverse effects or events refers to a reduction in the degree, duration, and/or frequency of adverse effects experienced by a subject and incidence in a group of subjects following administration of an agonist with about balanced affinity to GLP1R and GCGR compared to an agonist with unbalanced affinity for GLP1R and GCGR. Such reduction encompasses the prevention of some adverse effects that a subject would otherwise experience in response to an agonist with unbalanced affinity to GLP1R and GCGR. Such reduction also encompasses the elimination of adverse effects previously experienced by a subject following administration of an agonist with unbalanced affinity to GLP1R and GCGR. In some embodiments, “reducing,” or “reduction of” adverse effects encompass a reduction of gastrointestinal side effects wherein the adverse events are reduced to zero or undetectable levels. In other embodiments, adverse effect is reduced to level equivalent to untreated subjects but not completely eliminated. Morever, administration of analogs with unbalanced affinity toward GLP-1R or GCGR to a mammal may lead to the need for an excessively high dose to maximally activate the receptor with weaker sensitivity toward the ligand, thus leading to a potential for exceeding the biologically effective dose level for the other ligand and causing dose-related, undesired side effects.

This disclosure also provides methods for lowering and/or stabilizing the blood glucose of a mammal, the method comprising administering a pharmaceutical dosage formulation comprising a modified peptide of SEQ ID NOS. 1-29 (and/or a derivative thereof), preferably a modified peptide with about equal affinity for GLP-1R, GCGR and/or GIP, to a mammal, wherein the method reduces the incidence of, or the severity of, one of more adverse events as compared to an agonist with unbalanced affinity for GLP-1R, GCGR and/or GIP (e.g., semaglutide), the adverse events being selected from nausea, vomiting, diarrhea, abdominal pain and constipation, upon administration to a mammal. In some embodiments, such methods, as compared to a method in which an approximate equimolar dosage of semaglutide is administered, result in lower blood glucose (10, 20, 30, 40, 50, 60, 70, 80, 90, or 100% lower, preferably at least about 50% lower) at approximately 48 or 96 hours following administration, lower blood glucose at approximately 72 hours following administration (optionally at least about any of 10, 20, 30, 40, 50, 60, 70, 80, 90, or 100% lower, preferably at least about 100% lower), and/or, lower blood glucose at approximately 120 hours following administration (optionally at least about any of 10, 20, 30, 40, 50, 60, 70, 80, 90, or 100% lower, preferably at least about 100% lower); induces whole-body weight loss and/or induces liver weight loss; a lower Cmax (optionally about 10, 20, 30, 40, 50, 60, 70, 80, 90, or 100% lower, preferably at least about 50% lower), approximately equal or greater Tₘₐₓ (optionally about 10, 20, 30, 40, 50, 60, 70, 80, 90, or 100% lower, preferably at least about 100% greater Tₘₐₓ), a similar AUC_{(0-inf)} (optionally at least about any of 50, 60, 70, 80, 90, 95, 100% thereof, preferably at least about 80-90% thereof, such as about 85-93% thereof), approximately equal or greater T_{1/2(hr)} (optionally at least about any of 10, 20, 30, 40, 50, 60, 70, 80, 90, or 100% lower, preferably at least about 50 or 75% thereof, or about 50-75% thereof); a prolonged MRT (hr) (optionally prolonged by at least about any of 10, 20, 30, 40, 50, 60, 70, 80, 90, or 100%, preferably at least about 25%); a protracted PK/PD profile; equal or greater glucoregulatory effects; greater whole-body weight loss (optionally about twice the whole-body weight loss); lower body fat mass (optionally at least about any of 10, 20, 30, 40, 50, 60, 70, 80, 90, or 100% lower, preferably at least about 100% lower); greater loss in body weight by approximately 14 days following administration of the dosage formulation (optionally at least about any of 10, 20, 30, 40, 50, 60, 70, 80, 90, or 100% greater, preferably at least about 15% greater); greater loss in body weight by approximately 20-28 days following administration of the dosage formulation (optionally at least about any of 10, 20, 30, 40, 50, 60, 70, 80, 90, or 100% greater, preferably at least about 25% greater); and/or, weight loss in an obese mammal sufficient to return the weight of the mammal to the normal weight range of a lean normal mammal; and/or, when the method is for treating NASH, increased whole-body weight reduction, liver weight loss, improved NAS score, improved hepatosteatosis, improved ballooning, improved col1A1 staining, improved ALT, improved liver TG/TC, and improved plasma triglycerides (TG)/total cholesterol (TC).

In some embodiments, this disclosure provides pharmaceutical dosage formulations comprising an agonist peptide product and about 0.025-0.075% (w/w) polysorbate 20, about 0.2-0.5% (w/w) arginine, about 3-6% (w/w) mannitol in deionized water (pH 7.7±0.1). In preferred embodiments, the pharmaceutical dosage formulation comprises at least one modified peptide, about 0.050% (w/w) polysorbate 20 (PS-20), about 0.348% (w/w) arginine, and about 4.260% (w/w) mannitol in deionized water (pH 7.7±0.1). In some embodiments, the formulation can be modified to achieve its critical micelle concentration (CMC). In some embodiments, the concentration of PS-20 (i.e., 0.050% or 0.5 mg/ml) in the formulation can be raised to achieve the CMC and avoid a hazy appearance of the solution when stored at +2-8° C. In some embodiments, this can be achieved by including at least about 0.66 mg of PS-20 per mg of the peptide. In some embodiments, the formulation can substitute PS-20 with polysorbate 80 (PS-80, Tween 80) in an amount of at least about 1.03 mg of polysorbate 80 (PS-80, Tween 80) per mg of peptide to achieve the CMC.

In some embodiments, this disclosure provides pharmaceutical dosage formulations configured for administering to the mammal the agonist peptide product at less than about 0.72 mg/kg/dose, optionally from about 0.001 to 0.72 mg/kg/dose. In some embodiments, the pharmaceutical dosage formulation is configured to administer less than 0.36 mg/kg/dose of the agonist peptide product to the mammal. In some embodiments, the methods comprise administering between 0.001-0.3 mg/kg/dose, optionally about 0.007 mg/kg, or about 0.014 mg/kg or about 0.03 mg/kg, or about 0.07 mg/kg, or about 0.18 mg/kg/dose or about 0.25 mg/kg/dose. In some embodiments, the pharmaceutical dosage formulation can be configured to administer between about 0.05 to about 20 mg per week; optionally 0.1 to about 10 mg per week or optionally about 1 to about 7 mg per week; or optionally about 1 to 5 mg per week. In some embodiments, the pharmaceutical dosage formulation is configured to be administered to the mammal once weekly for up to six weeks. In some embodiments, this disclosure provides pharmaceutical dosage formulations configured such that the time to reach a therapeutic dose is about four weeks or less. In some embodiments, the therapeutic dose exhibits a Cₘₐₓ of from about 10 to about 2000 ng/ml; a Tₘₐₓ of from about 10 to about 168 hours; and/or, an AUC₀₋₁₆₈ of from about 1,000 to 100,000 h*ng/mL. In some embodiments, the agonist peptide may be repeatedly administered to achieve a plasma a concentration of about 5 to 1000 ng/ml or about 50 ng/ml, or about 150 ng/ml, or about 250 ng/ml or about 500 ng/ml.

In some embodiments, this disclosure provides the methods described herein that comprise administering to the mammal the agonist peptide product at less than about at less than about 0.72 mg/kg/dose, optionally from about 0.001 mg/kg/dose to less than about 0.36 mg/kg/dose, or optionally about 0.36 mg/kg/dose. In preferred embodiments of such methods, less than about 0.36 mg/kg/dose is administered to the mammal. In some embodiments, each dose is administered about once per week or once every two weeks, optionally for at least one month; optionally wherein each dose comprises about the same about of agonist peptide product. In some embodiments, such methods comprise administering about 0.72 mg/kg/dose once followed by one or more subsequent doses of from about 0.001 mg/kg/dose to about 0.36 mg/kg/dose. In some embodiments, the methods comprise administering between 0.001-0.30 mg/kg/dose, optionally about 0.007 mg/kg, or about 0.014 mg/kg or about 0.03 mg/kg, or about 0.07 mg/kg, or about 0.18 mg/kg/dose or about 0.25 mg/kg/dose. In some embodiments, the pharmaceutical dosage formulation can be configured to administer between about 0.05 to about 20 mg per week; optionally 0.1 to about 10 mg per week or optionally about 1 to about 7 mg per week; or optionally about 1 to 5 mg per week.

In preferred embodiments, such methods comprise administering the pharmaceutical dosage formulation subcutaneously. In some embodiments, such methods comprising administering the pharmaceutical dosage formulation to a mammal at about 0.03 to 0.25 mg/kg/dose exhibits a Cmax of from about 50 to about 1000 ng/ml; a Tmax of from about 10 to about 96 hours; and/or, an AUC0-168 of from about 1,000 to 80,000 h*ng/mL, or in some embodiments 5,000 to 80,000 h*ng/mL. In some such methods, the time to reach a therapeutic dose is about four weeks or less. In some embodiments, the therapeutic dose exhibits a Cmax of from about 50 to about 700 ng/ml; a Tmax of from about 10 to about 72 hours; and/or, an AUC0-168 of from about 2,000 to 70,000 h*ng/mL, or in some embodiments 6,000 to 70,000 h*ng/mL.

In some embodiments, the methods disclosed herein do not comprise a treatment initiation phase. In other words, the first administered dose is therapeutic without the need to titrate to avoid adverse gastrointestical side effects. For instance, in some embodiments, the method can comprise administering a first one or more doses (the treatment initiation phase) of a peptide of this disclosure, followed by subsequent second one or more and higher doses of the peptide, each of the first and second doses being administered for one or more weeks. In some embodiments, the first dose(s) and the second dose(s) can be followed by one or more third doses that can be higher than the second dose(s). The switch from the first dose, the second dose, and the third dose can be made on a weekly basis. For instance, if it appears the first dose has not induced lower blood glucose and/or weight loss after one or more weeks, the second higher dose can then be administered for one or more weeks followed by an analysis of the effects of the second dose(s). If the beneficial effects are observed (e.g., lower blood glucose and/or body weight), the second dose can continue to be administered. If the beneficial effects are not observed, the third dose may be administered for one or more weeks, followed by a determination of beneficial effects. This cycle of dosing and analysis can be repeated as appropriate, provided adverse events are not observed with each dose. In some embodiments, the subsequent second one or more and higher doses of the peptide can be administered because glycemic control (e.g., decreased blood glucose) was not achieved after about four weeks of administration of the first one or more doses. In some embodiments, the first one or more doses can be administered without the intention to produce a therapeutic effect (e.g., decreased blood glucose and/or weight loss). In some embodiments, however, the methods can be carried out without including the treatment initiation phase.

In some embodiments, the methods can be a first line indication for blood glucose control and/or weight loss in a human being, meaning that it is the first and sole active agent administered to the patient for the purpose of controlling blood glucose and/or inducing weight loss in the human being. In some embodiments, the methods disclosed herein can include an adjunct treatment of diet and/or exercise. In such embodiments, the human being can be administered the pharmaceutical dosage and provided with instructions regarding diet and/or exercise that can enhance the beneficial effects of the pharmaceutical dosage. In some embodiments, the human being to whom the pharmaceutical dosage is administered has type 2 diabetes mellitus. In some embodiments, the human being can exhibit established cardiovascular disease, with or without type 2 diabetes mellitus.

In some embodiments, the pharmaceutical dosage is administered about weekly. In some embodiments, the pharmaceutical dosage is administered to the human being about weekly from about 2 weeks to about 8 weeks, or longer. In some embodiments, the pharmaceutical dosage administered to the human being as a weekly dose for about 4 to about 8 weeks, optionally about 6 weeks, as compared to administration of an approximate equimolar dosage of semaglutide results in greater whole-body weight loss at about 1 week, about 2 weeks, about 3 weeks, about 4 weeks, about 5 weeks, about 6 weeks, or about 7 weeks following administration to the human being. In some embodiments, the pharmaceutical dosage is administered on about days 1, 8, 15, 22, 29, and 36. In some embodiments, the methods can include administration to the human being of a single dose, as compared to administration of an approximate equimolar dosage of semaglutide, results in lower blood glucose at about 1 day, about 2 days, about 3 days, about 4 days, about 5 days, about 6 days or about 7 days following administration. In some embodiments, the methods can include administration to human being of a weekly dose for about 4 to about 8 weeks, optionally about 6 weeks, as compared to administration of an approximate equimolar dosage of semaglutide, results in greater whole-body weight loss at about 1 week, about 2 weeks, about 3 weeks, about 4 weeks, about 5 weeks, about 6 weeks or about 7 weeks following administration. In some embodiments, the methods can include administration to the human being of a single dose, as compared to administration of an approximate equimolar dosage of semaglutide, exhibits a lower Cₘₐₓ at about 1 day, about 2 days, about 3 days, about 4 days, about 5 days, about 6 days or about 7 days following administration. In some embodiments, the methods can include administering the pharmaceutical dosage to an adult human at from about 0.5 mg/dose, about 1.0 mg/dose, about 1.5 mg/dose, about 2.0 mg/dose, about 2.5 mg/dose, about 3.0 mg/dose, about 3.5 mg/dose, about 4.0 mg/dose, about 4.5 mg/dose, about 5.0 mg/dose, or about 5.5 mg/dose. In some embodiments, the pharmaceutical dosage can be administered about once per week or once every two weeks, optionally for at least one month; optionally wherein each dose comprises about the same amount of agonist peptide product. In some embodiments, the pharmaceutical dosage can be administered subcutaneously. In some embodiments, one or more of the doses can be administered via a first route (e.g., subcutaneously) and subsequently administered by a different route (e.g., orally). In some embodiments, the time to reach a therapeutic dose is about four weeks or less. In some embodiments, administration of the pharmaceutical dosage formulation exhibits a Cₘₐₓ of from about 400 to about 1300 ng/ml; a Tₘₐₓ of from about 10 to about 36 hours; and/or, an AUC₀₋₄₈ of from about 15,000 to 45,000 h*ng/mL. In preferred embodiments, the weight loss in the human being is at least 5%, at least 10%; or from about 1% to about 20%; or from about 5% to about 10% (w/w). In some embodiments, administration thereof to a mammal results weight loss in an obese mammal sufficient to return the human being the normal weight range of a lean normal human being. In some embodiments, administration to a human being with a body mass index (BMI) indicative of obesity (e.g., about 30 or higher) exhibit a decrease in body weight of about 5-20%, such as about 15%, for an appropriate time (e.g., after any of about two, four, eight, 10, 20, or 30-100 weeks, such as about any of 50, 60, or 70 weeks). In preferred embodiments, the weight loss in such human beings is significant (e.g., P<0.001, 95% confidence interval (CI)). In some preferred embodiments, within about four weeks, administration to a human being results in at least about a 2-5% reduction in body weight, and in some embodiments continues and/or stabilizes until administration ceases. In some embodiments, in addition to weight loss, administration can also improved cardiovascular risk factors including greater reductions in waist circumference, BMI, systolic and diastolic blood pressures, HbA1c, fasting plasma glucose, C-reactive protein, and/or fasting lipid levels, as well as in some embodiments physical functioning scores and quality of life. In some embodiments, the pharmaceutical dosage form is an aqueous formulation comprising one or more of polysorbate 20, Arginine, or Mannitol.

Aspects of this disclosure include but are not limited to:

a modified peptide comprising a parent peptide any one or more of SEQ ID NOs. 1-29; GGG Tri-Agonist (Eli Lilly); GIP/GLP Coagonist Peptide (Eli Lilly); GIP/GLP Coagonist Peptide II (Eli Lilly); C2816 (Medimmune), a GLP-1/cholecystokinin receptor-1 (CCK1) co-agonist; ZP3022 (Zealand), a GLP-1/gastrin co-agonist; GLP-1/xenin co-agonist (University of Ulster); GIP/xenin co-agonist (University of Ulster); and, GLP-1/gastrin/xenin tri-agonist (University of Ulster); NNC 9204-1177 (NN9277) (Novo Nordisk; GLP-1R/GCGR); LY3305677 (Eli Lilly; GLP-1R/GCGR); JNJ-54728518 (Janssen Pharmaceuticals; GLP-1R/GCGR); LY2944876/TT-401 (Transition Therapeutics; GLP-1R/GCGR); CPD86 (Eli Lilly; GLP-1R/GIPR); LY3298176 (Tirzepatide); (Eli Lilly; GLP-1R/GIPR); LY3437943 (Eli Lilly; GLP-1R/GCGR/GIPR); SAR438335 (Sanofi; GLP-1R/GIPR); ZP-I-98 (Zealand; GLP-1R/GIPR); ZP-DI-70 (Zealand; GLP-1R/GIPR); HM15211 (Hanmi Pharmaceuticals; GLP-1R/GIPR/GCGR); NN9423/MAR423 (Novo Nordisk/Marcadia; GLP-1R/GIPR/GCGR), PB-719 (PegBio, GLP-1R/GIPR); DD01 (D&D Pharma, GLP-1R/GIPR); or a peptide sharing at least about 80% identity with any such peptides; wherein the modified peptide is covalently attached through an amino acid to a surfactant;

the modified peptide of the previous aspect wherein the surfactant is a group of Formula I:

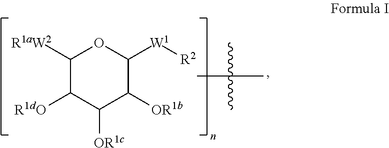

wherein R^{a} is independently, at each occurrence, a bond, H, a protecting group, a substituted or unsubstituted C₁-C30 alkyl group, a saccharide, a substituted or unsubstituted alkoxyaryl group, or a substituted or unsubstituted aralkyl group;

R^{1b}, Pv^{1c}, and R^{1d} are each, independently at each occurrence, a bond, H, a protecting group, a substituted or unsubstituted C₁-C₃₀ alkyl group, a substituted or unsubstituted alkoxyaryl group, or a substituted or unsubstituted aralkyl group;

W¹ is independently, at each occurrence, —CH₂—, —CH₂—O—, —(C═O), —(C═O)—O—, (C═O)—NH—, —(C═S)—, —(C═S)—NH—, or —CH₂—S—;

W² is —O—, —CH₂— or —S—;

R is independently, at each occurrence, a bond to U, H, a substituted or unsubstituted C₁-C₃₀ alkyl group, a substituted or unsubstituted alkoxyaryl group, or a substituted or unsubstituted aralkyl group, —NH, —S—, -triazolo-, —NH(C═O)—CH₂—, —(CH₂)ₘ-maleimide-;

n is 1, 2 or 3; and,

m is an integer of 1-10;

the modified peptide of any preceding aspect that is:
a) derived from SEQ ID NO:20 (Liraglutide):

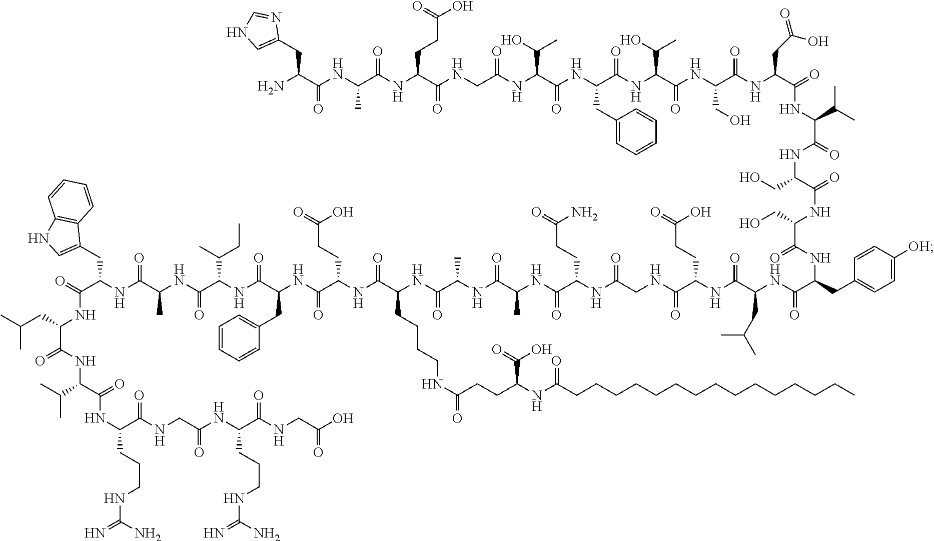

or,
b) derived from SEQ ID NO: 21 (Semaglutide);

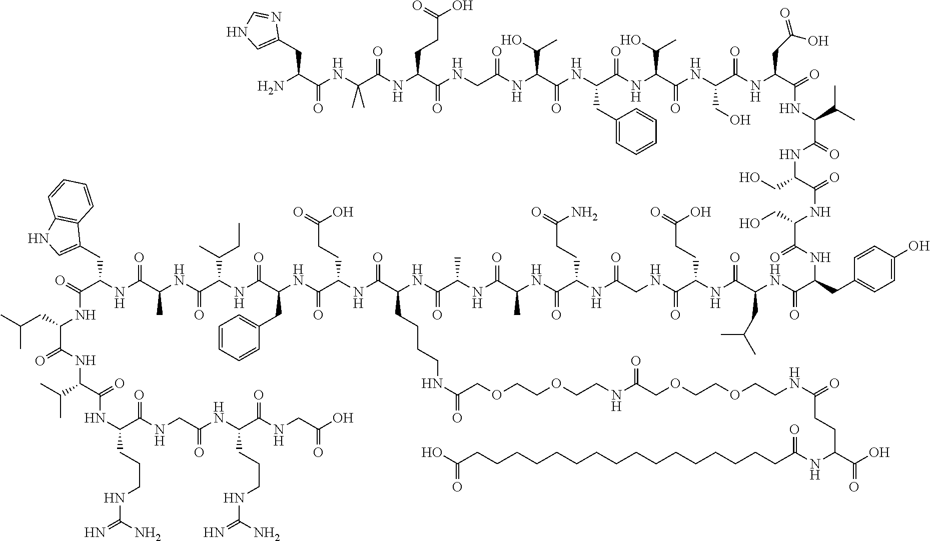

wherein X is Aib;

a pharmaceutical dosage formulation comprising a modified peptide of any preceding aspect and at least one pharmaceutically acceptable excipient;

a pharmaceutical dosage formulation comprising the modified peptide of any previous aspect, wherein the dosage is configured to improve control of blood glucose with reduction of one or more adverse events as compared to an unmodified version of the modified peptide;

a pharmaceutical dosage formulation of any preceding aspect, wherein the modified peptide has affinity for glucagon-like peptide 1 receptor (GLP-1R), gastric inhibitory polypeptide receptor (GIP-R) and/or glucagon receptor (GCGR);

a pharmaceutical dosage formulation of any preceding aspect, wherein weight loss is at least 5%, at least 10%; or from about 1% to about 20%; or from about 5% to about 10% (w/w);

a pharmaceutical dosage formulation of any preceding aspect, wherein the dosage is configured as a weekly dosage form, optionally configured for administration from about 2 weeks to about 8 weeks;

a pharmaceutical dosage formulation of any preceding aspect that is an aqueous formulation comprising one or more of polysorbate 20, Arginine, or Mannitol;

a pharmaceutical dosage formulation of any preceding aspect wherein administration thereof to a mammal, as compared to administration of an approximate equimolar dosage of an unmodified version of the modified peptide, results in: lower blood glucose at about 48 or 96 hours following administration, optionally wherein it is about 50% lower; lower blood glucose at about 72 hours following administration, optionally wherein it is about 100% lower; and/or, lower blood glucose at about 120 hours following administration;

a pharmaceutical dosage formulation of any preceding aspect configured to be administered to the mammal once weekly for at least, or up to six weeks;

a pharmaceutical dosage formulation of any preceding aspect configured such that the time to reach a therapeutic dose is about four weeks or less;

a pharmaceutical dosage formulation of any preceding aspect wherein the therapeutic dose exhibits a Cₘₐₓ of from about 400 to about 1300 ng/ml; a Tₘₐₓ of from about 10 to about 36 hours; and/or, an AUC₀₋₄₈ of from about 15,000 to 45,000 h*ng/mL;

a method for inducing weight loss in a mammal, the method comprising administering pharmaceutical dosage formulation of any preceding aspect to a mammal, wherein the method: reduces the incidence of one of more adverse events as compared to an unmodified version of the modified peptide, the adverse events being selected from nausea, vomiting, diarrhea, abdominal pain and constipation, upon administration to a mammal;

a method of any preceding aspect, wherein the pharmaceutical dosage is administered about weekly;

a method of any preceding aspect, wherein the pharmaceutical dosage is administered about weekly from about 2 weeks to about 8 weeks, or longer;

a method of any preceding aspect wherein each dose is administered about once per week or once every two weeks, optionally for at least one month; optionally wherein each dose comprises about the same about of modified peptide;

a method of any preceding aspect wherein the pharmaceutical dosage formulation is administered subcutaneously; and/or,

a method of any preceding aspect wherein the time to reach a therapeutic dose is about four weeks or less.

Other embodiments and/or aspects of this disclosure are also contemplated as will be understood by those of ordinary skill in the art.

Unless defined otherwise or clearly indicated otherwise by their use herein, all technical and scientific terms used herein have the same meaning as commonly understood by those of ordinary skill in the art to which this application belongs. As used in the specification and the appended claims, the word “a” or “an” means one or more. As used herein, the word “another” means a second or more. The acronym “aka” means also known as. The term “exemplary” as used herein means “serving as an example, instance or illustration”. Any embodiment or feature characterized herein as “exemplary” is not necessarily to be construed as preferred or advantageous over other embodiments or features. In some embodiments, the term “about” or “approximately” means within ±10% or 5% of the specified value. Whenever the term “about” or “approximately” precedes the first numerical value in a series of two or more numerical values or in a series of two or more ranges of numerical values, the term “about” or “approximately” applies to each one of the numerical values in that series of numerical values or in that series of ranges of numerical values. Ranges may be expressed herein as from about one particular value, and/or to about another particular value. When such a range is expressed, another aspect includes from the one particular value and/or to the other particular value. Similarly, when values are expressed as approximations, by use of the antecedent about or approximately, it will be understood that the particular value forms another aspect. It will be further understood that the endpoints of each of the ranges are significant both in relation to the other endpoint, and independently of the other endpoint. Ranges (e.g., 90-100%) are meant to include the range per se as well as each independent value within the range as if each value was individually listed. Optional or optionally means that the subsequently described event or circumstance can or cannot occur, and that the description includes instances where the event or circumstance occurs and instances where it does not. All publications, patents, and patent applications mentioned in this specification are herein incorporated by reference in their entireties to the same extent as if each individual publication, patent, or patent application was specifically and individually indicated to be incorporated by reference.

Certain embodiments are further described in the following examples. These embodiments are provided as examples only and are not intended to limit the scope of the claims in any way.

### EXAMPLES

### Example 1. Peptide Synthesis

A. 1-Alkyl β-d-glucuronic Acids. General Oxidation Method.

To a solution of 1-dodecyl β-d-glucopyranoside [2.0 g, 5.74 mmol] in 20 mL of acetonitrile and 20 mL of deionized water is added (diacetoxyiodo)benzene [4.4 g, 13.7 mmol] and TEMPO [0.18 g, 1.15 mmol]. The resulting mixture is stirred at room temperature until reaction completion (by 20 h). The reaction mixture is diluted with water and lyophilized to dryness to give crude product as a white powder of sufficient purity for direct use in coupling to the peptide Lys side chain (1.52 g, 73%). In a like manner the other 1-alkyl β-d-glucuronic or melibiouronic acids can be prepared and used to acylate the other peptide products described herein. The corresponding 1-substituted glucosides or melibosides are prepared using these procedures but substituting the appropriate chain length dicarboxylic starting materials to yield the desired chain length from the synthetic procedures, for example using hexadecanedioic acid, dodecanedioic acid, and the like, in place of octadecanedioic acid.

B. 18-(tertbutoxy)-18-oxooctadecanoic Acid

A suspension of octadecanedioic acid (40 g, 127 mmol) in toluene (500 ml) is heated at 95° C. under nitrogen. To the resulting solution, is added N,N-dimethylformamide di-tert-butylacetal (98 g, 434 mmol), dropwise over 3-4 hr. The reaction was stirred overnight at the same temperature, concentrated to dryness in vacuo and placed under high vacuum overnight. The resulting solid is suspended in CH₂Cl₂ (200 ml) with heat and sonication, and filtered at RT, washing with CH₂Cl₂. The filtrate (2) is concentrated to give the product as a solid (45 g, 86%) and used without further purification.

C. Tert-butyl 18-hydroxyoctadecanoate

A solution of 18-(tertbutoxy)-18-oxooctadecanoic acid (45 g, 121 mmol) in THF is cooled over an ice bath, under nitrogen and treated dropwise with borane dimethylsulfide complex (16 ml, 158 mmol). Vigorous gas evolution occurs over the first few milliliters of addition. After the addition, the mixture is slowly allowed to warm to RT and stirred overnight. The reaction is chilled over an ice bath, quenched with saturated sodium carbonate solution, diluted with ethyl acetate and washed with saturated sodium carbonate solution. The organic layer is concentrated in vacuo and placed under high vacuum overnight. The residue is dissolved in warm toluene (200 ml) and let stand for several hours at room temperature. The precipitated diol is removed by filtration through Celite, and the cake washed with toluene. The toluene solution is applied directly to a silica gel column and eluted with 10% ethyl acetate/hexane then 20% ethyl acetate/hexane, then 30% ethyl acetate/hexane and concentrated to give the product (24 g, 51%) as an oil which solidifies on standing. ¹H NMR (500 MHz, d₄-MeOH): δ=3.64 (m, 2H), 2.21 (t, 2H, J=9), 1.44 (s, 9H) 1.50-1.62 (m, 4H), 1.20-1.40 (m, 27H).

D. Tert-butyl 18-([1-beta-D-glucos-1-yl]oxy)octadecanoate

Tert-butyl 18-hydroxyoctadecanoate (46 g, 129 mmol) is dissolved in toluene (400 ml), concentrated in vacuo to circa 250 ml, and allowed to come to room temperature under nitrogen. To this solution was added HgO (yellow) (22.3 g, 103 mmol), HgBr₂ (37 g, 103 mmol), and acetobrom glucose with vigorous stirring. Stirring is continued overnight until alcohol was consumed and the mixture was filtered through Celite. The filtrate is treated with copper(II)triflate (1 g) and stirred for 1 hour until the orthoester (spot above product on TLC) was degraded. The reaction is then washed with water and the organic layer concentrated in vacuo. The residue is dissolved in methanol (500 ml) and treated with sodium methoxide (5.4 M in methanol) in 0.5 ml portions to bring the pH to 9 (spotting directly onto pH paper). The pH is checked every 0.5 hour and more sodium methoxide added as necessary to maintain the pH at 9. The reaction is complete in 4 hr. Acetic acid is added dropwise to bring the pH to 7, and the mixture concentrated in vacuo. The residue is loaded onto silica gel and purified by silica gel chromatography eluting with 5% methanol/CH₂Cl₂ then 10% methanol/CH₂Cl₂ to yield the product as a white solid (55 g, 82%). ¹H NMR (400 MHz, d₄-MeOH): δ=4.30 (d, 1H, J=7.6), 3.84 (m, 1H), 3.77 (d, 1H, J=9.6), 3.45-3.60 (m, 2H), 3.36 (t, 1H, J=9.2), 3.21 (t, 1H, J=8.4), 2.20 (t, 2H, J=7.2), 1.50-1.67 (m, 4H), 1.43 (s, 9H), 1.43-1.33 (m, 2H), 1.28 (br s, 24H)

E. Tert-butyl 18-([1beta-D-glucuron-1-yl]oxy)octadecanoate

Tert-butyl 18-([1-beta-D-glucos-1-yl]oxy)octadecanoate (50 g, 96 mmol) is dissolved in dioxane (800 ml) in a 2000 ml 3-neck flask with mechanical stirring and cooled to 10° C. To the solution is added 2,2,6,6-tetramethyl-1-piperidinyloxy (TEMPO) (150 mg, 0.96 mmol) and KBr (1.14 g, 9.6 mmol). Dropping funnels containing saturated Na₂CO₃ solution (300 ml) and 13% NaOCl solution (120 ml) sre fixed to the flask. The carbonate solution is started on a rapid drip and the NaOCl added at a slow drip (ca. 1 drop/second). After 100 ml of carbonate is been added, the pH is checked and more added as necessary to maintain ca. pH 10. The temperature is maintained at 10° C. to 15° C. throughout. After 3 hr. starting material remains so more NaOCl (10 ml) is added rapidly. After 0.5 hr. the reaction is quenched with methanol (10 ml). The mixture is poured into a 4000 ml Erlenmeyer flask, submerged in an ice bath and adjusted to pH 3 with 6 N HCl. The mixture is diluted with ethyl acetate and washed with 1 N HCl and 2× with distilled water allowing the layers to separate on the final wash. The organic layer is concentrated in vacuo to give the product as a white foam (38 g, 74%).

### Example 2, Modified Peptides—In Vitro Assays

Cellular assays can be carried out using standard cellular assays (DiscoveRx, LeadHunter assays) using readout of cAMP stimulation or arrestin activation. Compounds are weighed precisely and shipped to DiscoverX (Fremont, CA) for dilution and assay. The assay used was for the glucagon (human, cloned into CHO cells) GIP-R ((human, cloned into CHO cells) and GLP-1 (human, cloned into CHO cells) receptors in cellular assays. Assays are carried out in the presence of 0.1% ovalbumin. Historically such assays have been carried out in the presence of 0.1% BSA, but for these compounds which bind very tightly to serum albumin (>99%) it can distort the results and make the compounds seem much less potent. Use of 0.1% ovalbumin can avoid this problem. The improvement seen upon use of ovalbumin can be seen as an indicator of relative tightness of serum albumin binding for the peptide.

### Example 3. In Vivo Effects on Glucose, Body Weight, and Fat Loss in Mice

A. In Vivo Assays Using Db/db Mice. BKS.Cg-m+/+ Leprdb/J (Jackson Labs stock number 000642) male (“db/db”) mice at the age of 7-9 weeks of age are used in these studies, and maintained using standard animal care procedures. Studies initiated after one-week acclimation to facility conditions. On the morning of study day 0, mice are weighed and fasted for 4 hrs. Blood glucose is measured by glucometer using standard procedures. Mice can be selected based on body weights and those with blood glucose levels ≥300 mg/dL (i.e., diabetic) randomly assigned into groups. Clinical observations are conducted at receipt, prior to randomization, and daily from Days 1 to 5. Body weights are measured and recorded at receipt, prior to randomization, and daily from Days 1 to 5. Food consumption is measured and recorded daily from Days 1 to 5. Blood samples for glucose analysis are collected pretest (Day-3) and at 0, 1, 4, 8, 24, 48, 72, 96 and 120 hours following the single dose of the indicated modified peptide on Day 1.
B. In Vivo Assays Using “DIO JAX” Mice. A suitable number of 18 week-old male C57BL/6J mice, fed a high fat diet (Research Diets D12492) from the age of 6 weeks can be used for these studies (transferred to Jackson in vivo research laboratory (Sacramento, CA)). Before study initiation, all mice are continued on the high fat diet (60% kcal; D12492) and acclimated for four weeks. On the morning of Study Day-1, baseline body composition is determined for each mouse via NMR analysis. On the morning of Study Day 0, pre-dose blood glucose measurements are taken via glucometer and the mice were dosed, with dose time recorded. Blood glucose measurements are taken at 1, 2, 4, 8, 10, and 24 hours post-dose. After study day 1, pre-dose blood glucose is measured on days 4, 7, 9, 11, 13, 17, 21 and 25. Body weights and clinical observations are recorded every 2 days. Food intake in all groups is measured daily, following dosing. First food intake measurement is on Study Day-1. One group is pair-fed to other groups. On Study Day 27, the mice are fasted for 5 hours and a glucose tolerance test (GTT) performed. All mice are intra-peritoneally (IP) dosed with a bolus of glucose (2 g/kg) and blood glucose assessed pre-dose and 15, 30, 60, 90, and 120 minutes post-dose. All blood glucose values are entered in the GTT Blood Glucose Log.

### Example 4. Murine Non-Alcoholic Steatohepatitis (NASH)

In a DIO-NASH mouse study of male C57BL/6J mice are fed the Amylin High Fat Diet with 40% fat (including trans-fat), 18% fructose, 2% cholesterol diet for 29+ weeks. All mice entering the experiment are pre-biopsied, stratified based on liver biopsy (only animals with fibrosis 1 or above and steatosis 2 or above are included) animals stratified into groups based on Col1a1 immunostaining. For a total of 12 weeks of QD dosing animal groups are: vehicle; a modified peptide of this disclosure (e.g., derived from any of SEQ ID NOs. 1-29), and controls. Body weight (BW) is measured daily for the entire study period, food intake daily for the first 14 days then weekly until study end. Terminal plasma is measured for ALT/AST/TG/TC levels. Terminal liver removal and sampling is carried out for pre to post NAFLD Activity Score (NAS; HE staining) including Fibrosis Stage (Picrosirius red, PSR). Terminal histology is carried out for steatosis, Col1a1 and galectin-3 quantitation. Terminal liver workup includes TG+TC (extraction and measurement). Terminal liver biopsies are set up in: 1) 4% PFA for histology, 2) fresh frozen liver for biochemistry, 3) fresh frozen liver for RNA extraction and RNAseq. Treatment with the modified peptides of this disclosure (i.e., pharmaceutical formulation comprising one or more modified peptides derived from any of SEQ ID NOs. 1-29) is shown to decrease body weight in the NASH mouse model, preferably rapidly and in a dose-responsive manner; result in a superior NAFLD activity score (NAS) reduction as compared to controls (e.g., NAS scores ≤3); induce a reduction in the fat content of the livers of mice to that of the lean normal range (e.g., to similar to that of chow-fed lean normal mice); beneficial effects on fibrosis, as measured by liver Col1A1 and Galectin-3 content, compared to controls, or NASH vehicle control; significantly lower terminal liver Col1A1 and Galectin-3 levels as compared to NASH vehicle control, and other controls; to beneficially effect mean liver CollAl and Galectin-3 levels; to normalize liver triglycerides (TG), total cholesterol (TC), and plasma ALT; result in significantly lower terminal plasma AST levels compared to NASH vehicle control as well as significantly lower terminal plasma ALT levels as compared to NASH vehicle control, and other controls; suppression of inflammatory and profibrotic gene expression, particularly in the stellate cells pathway responsible for fibrotic lesion development; beneficial modulation of genes affecting fat usage and transport, including carnitine palmitoyl-transferase la (CPT-1), glycerol-3-phosphate acyltransferase 4 (GPAT-4) (p<0.001), and sterol regulatory element binding transcription factor 1 (SREBTF-1) as compared to NASH vehicle control after correction for gene-wise multiple testing; suppression of stellate cell pathway pro-fibrosis gene expression; suppression of cell death gene expression; and/or, suppression of liver inflammation gene expression.

What is claimed is:

1. A modified peptide comprising a peptide of any one or more of SEQ ID NOS: 1-29; a modified peptide comprising a peptide sharing at least about 80% identity with any of SEQ ID NOS: 1-29; a modified peptide comprising a peptide selected from the group consisting of GGG Tri-Agonist, GIP/GLP Coagonist Peptide, GIP/GLP Coagonist Peptide II, C2816, a GLP-1/cholecystokinin receptor-1 (CCK1) co-agonist, ZP3022, a GLP-1/gastrin co-agonist, GLP-1/xenin co-agonist, GIP/xenin co-agonist, GLP-1/gastrin/xenin tri-agonist, NNC 9204-1177 (NN9277), LY3305677, JNJ-54728518, LY2944876/TT-401, CPD86, LY3298176 (Tirzepatide), LY3437943, SAR438335, ZP-I-98, ZP-DI-70, HM15211, NN9423/MAR423, PB-719, and DD01; wherein the modified peptide is covalently attached through an amino acid to a non-ionic glycolipid surfactant.

2. The modified peptide of claim 1 wherein the non-ionic glycolipid surfactant is a group of Formula I: 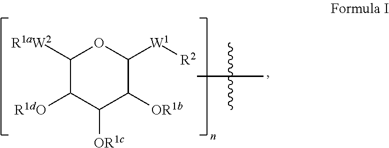
wherein R^{a} is independently, at each occurrence, a bond, H, a protecting group, a substituted or unsubstituted C₁-C30 alkyl group, a saccharide, a substituted or unsubstituted alkoxyaryl group, or a substituted or unsubstituted aralkyl group;
R^{1b}, Pv^{1c}, and R^{1d} are each, independently at each occurrence, a bond, H, a protecting group, a substituted or unsubstituted C₁-C₃₀ alkyl group, a substituted or unsubstituted alkoxyaryl group, or a substituted or unsubstituted aralkyl group;
W¹ is independently, at each occurrence, —CH₂—, —CH₂—O—, —(C═O), —(C═O)—O—, (C═O)—NH—, —(C═S)—, —(C═S)—NH—, or —CH₂—S—;
W² is —O—, —CH₂— or —S—;
R is independently, at each occurrence, a bond to U, H, a substituted or unsubstituted C₁-C₃₀ alkyl group, a substituted or unsubstituted alkoxyaryl group, or a substituted or unsubstituted aralkyl group, —NH, —S—, -triazolo-, —NH(C═O)—CH₂—, —(CH₂)m-maleimide-;
n is 1, 2 or 3; and,
m is an integer of 1-10.

3. The modified peptide of claim 1 wherein the peptide sequence is SEQ ID NO: 24 or SEQ ID NO: 25, wherein the surfactant is conjugated at amino acid residue 20.

4. A pharmaceutical dosage formulation comprising a modified peptide of any preceding claim and at least one pharmaceutically acceptable excipient.

5. A pharmaceutical dosage formulation comprising the modified peptide of any preceding claim, wherein the dosage is configured to improve control of blood glucose with reduction of one or more adverse events as compared to an unmodified or parent version of the modified peptide.

6. A pharmaceutical dosage formulation of any preceding claim, wherein the modified peptide has affinity for glucagon-like peptide 1 receptor (GLP-1R), gastric inhibitory polypeptide receptor (GIP-R) and/or glucagon receptor (GCGR).

7. The pharmaceutical dosage formulation of any preceding claim, wherein weight loss is at least 5%, at least 10%; or from about 1% to about 20%; or from about 5% to about 10% (w/w).

8. The pharmaceutical dosage formulation of any preceding claim, wherein the dosage is configured as a weekly dosage form, optionally configured for administration from about 2 weeks to about 8 weeks.

9. The pharmaceutical dosage formulation of any preceding claim, administered to a mammal of a single dose, as compared to administration of an approximate equimolar dosage of unmodified peptide, exhibits a lower Cₘₐₓ at about 1 day, about 2 days, about 3 days, about 4 days, about 5 days, about 6 days or about 7 days following administration.

10. The pharmaceutical dosage formulation of any preceding claim configured to be administered to the mammal once weekly for at least, or up to six weeks.

11. The pharmaceutical dosage formulation of any preceding claim configured such that the time to reach a therapeutic dose is about four weeks or less.

12. A method for lowering the blood glucose of a mammal, the method comprising administering a modified peptide and/or pharmaceutical dosage formulation of any preceding claim to a mammal, wherein the method reduces the incidence of one of more adverse events as compared to an unmodified version of the modified peptide, the adverse events being selected from nausea, vomiting, diarrhea, abdominal pain and constipation, upon administration to a mammal.

13. A method for inducing weight loss in a mammal, optionally wherein the mammal is overweight or obese, the method comprising administering pharmaceutical dosage formulation of any preceding claim to a mammal, wherein the method: reduces the incidence of one of more adverse events as compared to an unmodified version of the modified peptide, the adverse events being selected from nausea, vomiting, diarrhea, abdominal pain and constipation, upon administration to a mammal.

14. The method of any preceding claim, wherein the pharmaceutical dosage is administered about weekly.

15. The method of any preceding claim, wherein the pharmaceutical dosage is administered about weekly from about 2 weeks to about 8 weeks, or longer.

16. The method of any preceding claim wherein each dose is administered about once per week or once every two weeks, optionally for at least one month; optionally wherein each dose comprises about the same about of modified peptide.

17. The method of any preceding claim wherein the pharmaceutical dosage formulation is administered subcutaneously.

18. The method of any preceding claim wherein the time to reach a therapeutic dose is about four weeks or less.


SEQUENCE LISTING (29 sequences)

SEQ ID NO: 1 - protein, 30 aa, Artificial sequence
  Synthetic peptide
HAQGTFTSDYSKYLDEKRAKEFVQWLMNTZ

SEQ ID NO: 2 - protein, 37 aa, Artificial sequence
  Synthetic Peptide
HSQGTFTSDYSKYLDSRRAQDFVQWLMNTKRNRNNIA

SEQ ID NO: 3 - protein, 41 aa, Artificial sequence
  Synthetic Peptide
  MOD_RES (31)..(31): NH2
  misc_feature (41)..(41): Xaa can be any naturally occurring amino acid
HSDQGTFTSDLSKQKESKAAQDFIEWLKAGGPSSGAPPPSX

SEQ ID NO: 4 - protein, 30 aa, Artificial sequence
  Synthetic Peptide
HSQGTFTSDKSEYLDSERARDFVAWLEAGG

SEQ ID NO: 5 - protein, 29 aa, Artificial sequence
  Synthetic Peptide
  MOD_RES (2)..(2): Aib
HXQGTFTSDKSKYLDERAAQDFVQWLLDT

SEQ ID NO: 6 - protein, 35 aa, Artificial sequence
  Synthetic peptide
  MOD_RES (2)..(2): Aib
HXQGTFTSDYSKYLDERAAKDFIEWLEKGSGSGGA

SEQ ID NO: 7 - protein, 40 aa, Artificial sequence
  Synthetic peptide
  MOD_RES (2)..(2): Aib
  MOD_RES (20)..(20): Aib
YXQGTFTSDYSIYLDKQAAXQFVNYLLAGGPSSGAPPPSK

SEQ ID NO: 8 - protein, 39 aa, Artificial sequence
  Synthetic peptide
  MOD_RES (2)..(2): Aib
  MOD_RES (13)..(13): Aib
  MOD_RES (20)..(20): conjugation to the epsilon-amino group of the side-chain
  MOD_RES (22)..(22): Phe or 1-Nal
YXEGTFTSDYSIXLDKIAQKAXVQWLIAGGPSSGAPPPS

SEQ ID NO: 9 - protein, 38 aa, Artificial sequence
  Synthetic peptide
  MOD_RES (2)..(2): Aib
  MOD_RES (12)..(12): Aib
  MOD_RES (21)..(21): Phe or 1-Nal
YXGTFTSDYSIXLDKIAQKAXVQWLIAGGPSSGAPPPS

SEQ ID NO: 10 - protein, 29 aa, Artificial sequence
  Synthetic peptide
  MOD_RES (16)..(16): Aib
YSHGTFTSDVSKYLDXKKAAEFVAWLLEE

SEQ ID NO: 11 - protein, 40 aa, Artificial squence
  Synthetic peptide
  MOD_RES (16)..(16): Aib
HAQGTFLSDYSKYLDXKKAQEFVEWLLKTGPSSGAPPKSK

SEQ ID NO: 12 - protein, 40 aa, Artificial sequence
  Synthetic peptide
  MOD_RES (16)..(16): Aib
YSQGTFTSDYSKYLDXKKAQEFVEWLLKTGPSSGAPPKSK

SEQ ID NO: 13 - protein, 34 aa, Artificial sequence
  Synthetic peptide
  MOD_RES (2)..(2): Aib
HXQGTFTSDYSKYLDEKKAKEFVEWLLEGGPSSG

SEQ ID NO: 14 - protein, 34 aa, Artificial sequence
  Synthetic peptide
  MOD_RES (2)..(2): Aib
HXQGTFTSDYSKYLDEKKAKEFVEWLLSGGPSSG

SEQ ID NO: 15 - protein, 39 aa, Artificial sequence
  Synthetic peptide
  MOD_RES (2)..(2): Aib
  MOD_RES (3)..(3): Gln or His
  MOD_RES (6)..(6): MePhe or MeF(2F)
  MOD_RES (13)..(13): Leu or aMeL
  MOD_RES (17)..(17): any amino acid with a functional group available for conjugation to a C16-C22 fatty acid
  MOD_RES (20)..(20): Aib, Gln or His
YXXGTXTSDYSIXLDKXAQXAFIEYLLEGGPSSGAPPPS

SEQ ID NO: 16 - protein, 33 aa, Artificial sequence
  Synthetic peptide
  misc_feature (2)..(4): Xaa can be any naturally occurring amino acid
  misc_feature (7)..(7): Xaa can be any naturally occurring amino acid
  misc_feature (11)..(15): Xaa can be any naturally occurring amino acid
  misc_feature (17)..(18): Xaa can be any naturally occurring amino acid
  misc_feature (20)..(33): Xaa can be any naturally occurring amino acid
RXXXGTXTSDXXXXXDXXAXXXXXXXXXXXXXX

SEQ ID NO: 17 - protein, 29 aa, Artificial sequence
  Synthetic peptide
HSQGTFTSDYSKYLDSRRAQDFVQWLMNT

SEQ ID NO: 18 - protein, 42 aa, Artificial sequence
  Synthetic peptide
YAEGTFISDYSIAMDKIHQQDFVNWLLAQKGKKNDWKHNITQ

SEQ ID NO: 19 - protein, 31 aa, Artificial sequence
  Synthetic peptide
HAEGTFTSDVSSYLEGQAAKEFIAWLVKGRG

SEQ ID NO: 20 - protein, 31 aa, Artificial sequence
  Synthetic peptide
HAEGTFTSDVSSYLEGQAAKEFIAWLVRGRG

SEQ ID NO: 21 - protein, 31 aa, Artificial sequence
  Synthetic peptide
  MOD_RES (2)..(2): Aib
HXEGTFTSDVSSYLEGQAAKEFIAWLVRGRG

SEQ ID NO: 22 - protein, 32 aa, Artificial sequence
  Synthetic peptide
HGEGTFTSDVSSYLEEQAAKEFIAWLVKGGGZ

SEQ ID NO: 23 - protein, 29 aa, Artificial sequence
  Synthetic peptide
HSQGTFTSDYSKYLDSRRAQDFVQWLMNT

SEQ ID NO: 24 - protein, 33 aa, Artificial sequence
  Synthetic peptide
HGDGSFSDEMNTILDNLAARDFINWLIQTKITD

SEQ ID NO: 25 - protein, 39 aa, Artificial sequence
  Synthetic peptide
  MOD_RES (2)..(2): Aib
  MOD_RES (13)..(13): Aib
YXEGTFTSDYSIXLDKIAQKAFVQWLIAGGPSSGAPPPS

SEQ ID NO: 26 - protein, 39 aa, Artificial sequence
  Synthetic peptide
  MOD_RES (2)..(2): Aib
  MOD_RES (20)..(20): Aib
  MOD_RES (34)..(34): Aib
HXHGTFTSDLSKLKEEQRQXEFIEWLKALGPPSXKPPPK

SEQ ID NO: 27 - protein, 39 aa, Artificial sequence
  Synthetic peptide
  MOD_RES (2)..(2): Aib
  MOD_RES (13)..(13): Leu or AlphaMeL
  MOD_RES (17)..(17): Aib
  MOD_RES (19)..(19): Gln or Ala
  MOD_RES (20)..(20): Aib, AlphaMeK, Gln or His
  MOD_RES (28)..(28): Glu or Ala
  MOD_RES (29)..(29): Gly or Aib
  MOD_RES (34)..(34): Gly or Aib
YXQGTFTSDYSIXLDKKAXXAFIEYLLXXGPSSXAPPPS

SEQ ID NO: 28 - protein, 30 aa, Artificial sequence
  Synthetic peptide
  MOD_RES (1)..(1): His, 4-imidazoacetyl (CA), or Tyr
  MOD_RES (2)..(2): Gly, Alpha-methyl-glutamic acid or Aib
  MOD_RES (3)..(3): Glu, AlphaMeGlu or Aib
  MOD_RES (7)..(7): Thr or Ile
  MOD_RES (10)..(10): Leu, Tyr, Lys, Cys or Val
  MOD_RES (12)..(12): Lys, Ser or Ile
  MOD_RES (13)..(13): Gln, Tyr, Ala or Cys
  MOD_RES (14)..(14): Leu, Met or Tyr
  MOD_RES (15)..(15): Cys, Asp, Glu or Leu
  MOD_RES (16)..(16): Gly, Glu or Ser
  MOD_RES (17)..(17): Gln, Arg, Ile, Glu, Cys or Lys
  MOD_RES (18)..(18): Ala, Gln, Arg or His
  MOD_RES (19)..(19): Ala, Gln, Cys or Val
  MOD_RES (20)..(20): Lys, Gln, or Arg
  MOD_RES (21)..(21): Glu, Gln, Leu, Cys or Asp
  MOD_RES (23)..(23): Ile or Val
  MOD_RES (24)..(24): Ala, Gln, Cys Asn, Asp, or Glu
  MOD_RES (27)..(27): Val, Leu, Lys, or Met
  MOD_RES (28)..(28): Cys, Lys, Ala, Asn, or Asp
  MOD_RES (29)..(29): Cys, Gly, Gln, Thr, Glu or His
  MOD_RES (30)..(30): Cys, Gly, Lys, or HIs or is absent
XXXGTFXSDXSXXXXXXXXXXFXXWLXXXX

SEQ ID NO: 29 - protein, 30 aa, Artificial sequence; identical to SEQ ID NO: 4
  Synthetic Peptide